(19) United States
(12) Patent Application Publication
(10) Pub. No.: US 2020/0316087 A1
(43) Pub. Date: Oct. 8, 2020

(54) NOVEL COMBINATION THERAPY FOR ANXIETY DISORDERS, EPILEPSY, AND PAIN

(71) Applicants: UWM Research Foundation, Inc., Milwaukee, WI (US); The Research Foundation for the State University of New York, Amherst, NY (US)

(72) Inventors: James M. Cook, Milwaukee, WI (US); Veera Venkata Naga Phani Babu Tiruveedhula, Milwaukee, WI (US); Jun-Xu Li, East Amherst, NY (US)

(21) Appl. No.: 16/652,914

(22) PCT Filed: Oct. 3, 2018
(86) PCT No.: PCT/US2018/054248

§ 371 Date: Apr. 1, 2020

Related U.S. Application Data
(60) Provisional application No. 62/567,426, filed on Oct. 3, 2017.

(51) Int. Cl. A61K 31/5517; A61K 31/5513; A61P 25/22; A61P 25/08; A61P 23/00

(52) U.S. Cl. CPC A61K 31/5517; A61K 31/5513; A61P 23/00; A61P 25/08; A61P 25/22

(57) ABSTRACT

Combination therapy with a GABA_{A} agonist and a α1β2/3γ2 GABA inhibitor is for the treatment of pain, epilepsy, or depression with reduced GABA_{A} agonist-mediated adverse effects compared with GABA_{A} agonist therapy alone. Effective doses of α1β2/3γ2 GABA inhibitor reduce GABA_{A} agonist adverse effects without substantial inhibition of therapeutic efficacy.

### RELATED APPLICATIONS

The application claims the benefit of U.S. provisional application Ser. No. 62/567,426, filed Oct. 3, 2017, which is incorporated herein by reference.

### STATEMENT REGARDING FEDERALLY SPONSORED RESEARCH OR DEVELOPMENT

This invention was made with government support under R01 AAA016179 and ROT DA034806 awarded by the National Institutes of Health (NIH). The United States government has certain rights to this invention.

### TECHNICAL FIELD

The present disclosure relates to the compounds, compositions, and methods for use in treating a pain, epilepsy, or depression disorder and alleviating adverse effects mediated by GABA_{A} agonists.

### BACKGROUND

In the present era, combinational therapy has become one of the key treatment techniques in drug discovery and development processes because of the ability to treat many disease settings, including cancer, infectious diseases, cardiovascular diseases and central nervous system disorders (Cheng, G. et al. Front Microbial 2016, 7, 470; Lehar, J. et al. Nat Biotechnol 2009, 27, 659; Reinmuth, N. et al. Prog Tumor Res 2015, 42, 79; Zhang, H. H. et al. Cancer Chemother Pharmacol 2016, 78, 13). Recent scientific discoveries have increased the understanding of the pathophysiological processes that underlie these and other complex diseases. Furthermore, the impetus to develop new therapeutic approaches using combinations of drugs directed at multiple therapeutic targets to improve treatment response, minimize the development of resistance, or minimize adverse events as well as tolerance can be reapplied even to reposition earlier approved treatments. Consequently, combination therapy provides significant therapeutic advantages. Hence, there is growing interest in the development of combinations of new investigational drugs.

The blood-brain barrier is one of the major protective layers for the central nervous system, the most complex of human organs and determines our most unique human function, namely, consciousness (Domínguez, A. et al. Neuroscience Discovery 2013, 1, 3.). Gamma-aminobutyric acid (GABA) plays a vital role in the treatment of central nervous system disorders and is the major inhibitory neurotransmitter in the CNS. GABA receptors are heteromeric, and are divided into three main classes: (1) GABA_{A} receptors, which are members of the ligand-gated ion channel superfamily; (2) GABA_{B} receptors, which may be members of the G-protein linked receptor superfamily; and (3) GABA_{C} receptors, also members of the ligand-gated ion channel superfamily, but their distribution is confined to the retina. Benzodiazepine receptor ligands do not bind to GABA_{B} and GABA_{C} receptors. Since the first cDNAs encoding individual GABA_{A} receptor subunits were cloned, the number of known members of the mammalian family has grown to 21 including α, β, and γ subunits (6α, 4β, 4γ, 1β, 1ε, 1π, 1θ, and 3ρ).

A characteristic property of GABA_{A} receptors is the presence of a number of modulatory sites, one of which is the benzodiazepine (BZ) site. The benzodiazepine binding site is the most explored of the GABA_{A} receptor modulatory sites, and is the site through which benzodiazepine-based anxiolytic drugs exert their effect. Before the cloning of the GABA_{A} receptor gene family, the benzodiazepine binding site was historically subdivided into two subtypes, BENZODIAZEPINE1 and BENZODIAZEPINE2, on the basis of radioligand binding studies on synaptosomal rat membranes. The BENZODIAZEPINE1 subtype has been shown to be pharmacologically equivalent to a GABA_{A} receptor comprising the α1 subunit in combination with a β subunit and γ2. It has been shown that an α subunit, a β subunit, and a γ subunit constitute the minimum requirement for forming a fully functional benzodiazepine/GABA_{A} receptor.

Receptor subtype assemblies for BZ-sensitive GABA_{A} receptors include amongst others the subunit combinations α1β2/3γ2, α2β2/3γ2, α3β2/3γ2, and α5β2/3γ2. The α4β2/3γ2 and α6β2/3γ2 subtypes are termed benzodiazepine-insensitive receptors for they do not interact with classical benzodiazepines such as diazepam. Subtype assemblies containing an α1 subunit (α1β2/3γ2) are present in most areas of the brain and are thought to account for 40-50% of GABA_{A} receptors in rat brain. Subtype assemblies containing α2 and α3 subunits respectively are thought to account for about 25% and 17% of GABA_{A} receptors in rat brain, respectively. Subtype assemblies containing an α5 subunit (α5β2/3γ2) are expressed predominately in the hippocampus and cortex and are thought to represent about 5% of GABA_{A} receptors in the rat. Two other major populations are the α2β2/3γ2 and α3β2/3γ2 subtypes as stated above. Together these constitute approximately a further 35% of the total GABA_{A} receptor population. Pharmacologically this combination appears to be equivalent to the BENZODIAZEPINE2 subtype as defined previously by radioligand binding, although the BENZODIAZEPINE2 subtype may also include certain α5-containing subtype assemblies.

The present accepted pharmacology of agonists acting at the BZ binding site of GABA_{A} receptors suggests that α1 containing receptors mediate sedation, anticonvulsant activity, ataxia, anterograde amnesia, tolerance, and addiction. While α2 and/or α3 GABA_{A} receptors mediate anxiolytic activity, anticonvulsant activity, and antinociceptive activity. The α5 containing GABA_{A} receptors are involved in memory functions (U. Rudolph et al., Nature 1999, 401, 796; K. Low et al., Science 2000, 290, 131; McKernan Nature Neurosci. 2000, 3, 587; F. Crestani et al., Proc. Nat. Acad. Sci. USA 2002, 99, 8980; M. S. Chambers et al., J. Med. Chem. 2003, 46, 2227).

It is believed that agents acting selectively as benzodiazepine agonists at GABA_{A}/α2, GABA_{A}/α3, and/or GABA_{A}/α5 receptors possess desirable properties. Compounds which are modulators of the benzodiazepine binding site of the GABA receptors by acting as benzodiazepine agonists are referred to hereinafter as “GABA_{A} receptor agonists,” The GABA_{A}/α1-selective (α1β2/3γ2) agonists alpidem and zolpidem are clinically prescribed as hypnotic agents, suggesting that at least some of the sedation associated with known anxiolytic drugs which act at the Benzodiazepine 1 binding site is mediated through GABA_{A} receptors containing the α1 subunit. Recently, two studies have shown that the majority of addictive properties of diazepam are mediated by α1 subtypes (N. A. Ator et. al. J. Pharm. Exp. Thera. 2010, 332, 4; K. R. Tan et. al., Nature, 2010, 463, 769), It is also known that tolerance is due to an agonist response at α1 receptors and or the coupling of α1 receptors to α5 receptors (Van Rijnsoever et al. J Neurosci 2004, 24, 6785).

The most frequently prescribed medication for treatment of anxiety disorders (such as phobias, obsessive compulsive disorders) and seizure disorders are benzodiazepines such as alprazolam, clonazepam, diazepam, lorazepam and other benzodiazepine-based medications. However, these benzodiazepine-based medications have side effects such as drowsiness, sedation, motor incoordination, memory impairment, potentiation of effects of alcohol, tolerance and dependence, and abuse potential. Buspirone, tandospirone, and other serotonergic agents have been developed as anxiolytics with a potentially reduced profile of side effects. However, while these medications do show a reduced profile of side effects, they have other characteristics which make them less than ideal for treatment of anxiety disorders. In some cases, these agents cause anxiety before a therapeutic dose can be obtained or require dosing of the drug for several days before a therapeutic effect is seen. In addition SSRI's commonly cause sexual dysfunction. Development of anxiolytics devoid of sedation, ataxia, amnesia, tolerance and addiction represent an unmet need (Cook, J. M. et al. U.S. Pat. No. 7,119,196 B2, Oct. 10, 2006).

### SUMMARY

One aspect of the invention provides a method of treating a disorder selected from the group consisting of pain, epilepsy, and depression comprising administering to a subject in need thereof, a therapeutically effective amount of a GABA_{A} agonist, or a pharmaceutically acceptable salt thereof, and a α1β2/3γ2 GABA inhibitor, or a pharmaceutically acceptable salt thereof, in an amount effective to inhibit an adverse effect mediated by the GABA_{A} agonist.

Another aspect of the invention provides a method of inhibiting an adverse effect of a GABA_{A} agonist, the adverse effect being selected from the group consisting of tolerance to antinociception, drowsiness, and addiction, comprising administering to a subject in need thereof, an effective amount of a α1β2/3γ2 GABA inhibitor.

Another aspect of the invention provides a pharmaceutical combination comprising a GABA_{A} agonist, or a pharmaceutically acceptable salt thereof, and a α1β2/3γ2 GABA inhibitor, or a pharmaceutically acceptable salt thereof, for use in the treatment of a disorder selected from the group consisting of pain, epilepsy, and depression.

Another aspect of the invention provides a α1β2/3γ2 GABA inhibitor, or a pharmaceutically acceptable salt thereof, for use in the inhibition of an adverse effect mediated by a GABA_{A} agonist, the adverse effect being selected from the group consisting of tolerance to antinociception, drowsiness, and addiction.

Another aspect of the invention provides a use of a pharmaceutical combination of a GABA_{A} agonist, or a pharmaceutically acceptable salt thereof, and a α1β2/3γ2 GABA inhibitor, or a pharmaceutically acceptable salt thereof, for the preparation of a medicament for the treatment of pain, epilepsy, or depression.

Another aspect of the invention provides a use of α1β2/3γ2 GABA inhibitor, or a pharmaceutically acceptable salt thereof, for the preparation of a medicament for the inhibition of an adverse effect mediated by a GABA_{A} agonist, the adverse effect being selected from the group consisting of tolerance to antinociception, drowsiness; and addiction.

Another aspect of the invention provides a pharmaceutical composition comprising a GABA_{A} agonist, or a pharmaceutically acceptable salt thereof; a α1β2/3γ2 GABA inhibitor, or a pharmaceutically acceptable salt thereof; and a pharmaceutically acceptable carrier.

Another aspect of the invention provides a kit comprising a GABA_{A} agonist, or a pharmaceutically acceptable salt thereof; a α1β2/3γ2 GABA inhibitor, or a pharmaceutically acceptable salt thereof; and instructions for use.

Another aspect of the invention provides a pharmaceutical combination comprising a GABA_{A} agonist, or a pharmaceutically acceptable salt thereof, and a α1β2/3γ2 GABA inhibitor, or a pharmaceutically acceptable salt thereof, for use as a medicament.

### BRIEF DESCRIPTIONS OF THE DRAWINGS

FIG. 1 shows clinically available benzodiazepines.

FIG. 2 shows the structures of HZ-166, KRM-II-81, KRM-II-82, MY-III-080, KRM-II-18B and NS16085.

FIG. 3 shows α1-preferring antagonists.

FIG. 4A and FIG. 4B show the percent modulation of GABA-evoked current responses in voltage clamped Xenopus oocytes expressing recombinant GABA_{A} receptors. Each oocyte was injected with cRNA of indicated α subunit together with cRNA of β3 and γ2 subunits. GABA concentration is at the EC50 for each receptor subunit combination. Concentration of indicated modulatory is saturating (1-10 μM). The peak whole cell current response from application of GABA and modulator is reported as the percentage of the peak response to GABA alone (% GABA Response), Each value is the mean±standard deviation for 3 or more separate oocytes.

FIG. 5 shows the antinociceptive effects of midazolam during daily 10 mg/kg midazolam or the combined 10 mg/kg midazolam and 5.6 mg/kg 3-PBC/βCCt treatment in a rat model of complete Freund's adjuvant-induced inflammatory pain. The pain-like behavior was measured using von Frey filament test. Midazolam dose-effect curves were determined using a cumulative dosing procedure. The data from pain measures (gram) were converted to percentage of maximal possible effect. (N=6 per group).

FIG. 6 shows the dose-dependent attenuations of the antinociceptive effects of KRM-II-81 by Flumazenil using a rat model of inflammatory pain.

FIG. 7A, FIG. 7B, FIG. 7C and FIG. 7D show KRM-II-81 suppression of the hyper-excitation in a network of cortical neurons. Reversible potentiation of spontaneous neuronal activity (FIG. 7A-spiking, FIG. 7C-bursting) by removal of magnesium or by addition of 1 mM 4-aminopyridine (4AP) to external solution. Removal of magnesium produced an increase in spiking frequency (347±81% of control, p=0.04, n=8) and an increase in bursting frequency (443±128% of control, p=0.02, n=6). Addition of 1 mM 4-aminopyridine (4-AP) to the external solution primarily increased the frequency of spikes (327±46% of control, p=0.005, n=12) with smaller effect on frequency of bursts (120±21% of control, p>0.05, n=10). KRM-II-81 (3 μM, FIG. 7B) had no significant effect on spiking (n=7) in the network of cortical neurons bathed in normal magnesium containing external solution and produced small depression of spiking under conditions of reduced magnesium (0.1 mM Mg++, n=4, p>0.05). When the network was hyper-excited by removal of magnesium in external solution, the addition of 3 μM KRM-II-81 suppressed the frequency of spiking to 62.2±3.8% of control (p=0.03, n=7), A similar effect of 3 μM KRM-II-81 was observed in a neuronal network hyper-excited by addition of 1 mM 4AP where the frequency of spiking was reduced to 50.5±7.5% of control (p=0.004, n=7), KRM-II-81 (3 μM, FIG. 7D) had no significant effect on bursting in normal magnesium (n=7) and in reduced magnesium containing external solution (0.1 mM Mg++, n=4, p>0.05). When the network was hyper-excited by removal of magnesium 3 μM KRM-II-81 reduced the frequency of bursting to 54.8±7.1% of control, (p=0.06, n=5). A similar effect of 3 μM KRM-II-81 was observed in a neuronal network hyper-excited by addition of 1 mM 4AP where the frequency of bursting was reduced to 50.9±8.8% of control (p=0.03, n=6). The data were normalized to baseline activity and reported as mean±standard error of the mean (SEM). One parameter t-test to determine statistical difference (FIG. 7A and FIG. 7C); analysis of variance (ANOVA) with Dunnett's multiple comparison test was utilized to compare between group effects (FIG. 7B and FIG. 7D); P<0.05 was considered significant (asterisk). MEA recordings from a culture of rat E18 cortical neurons (DIV 19-25). All recordings were performed at 37° C.

FIG. 8 shows the comparative effects of HZ-166, KRM-II-81, KRM-II-82, MP-III-080 and diazepam against electroshock-induced convulsions in mice. Quantal data were analyzed by Fisher's Exact Probability test (*:p<0.05). Each point represents the effects in 10 mice. Baseline values across studies (effects of drug vehicle) was 94±2.5%

FIG. 9 shows the comparative effects of HZ-166, KRM-II-81, and diazepam against clonic convulsions induced by pentylenetetrazole (35 mg/kg, s.c.) and on motor performance on an inverted screen in rats, Each point represents the effect in groups of 5-8 rats. Quantal data were analyzed by Fisher's Exact Probability test (*:p<0.05). For motor scores, each point represents the mean±SEM in groups of 5 (diazepam, 3 mg/kg) or 8 (all other data) rats. Data were analyzed by ANOVA followed by Dunnett's test with * signifying statistically-significant separation from vehicle control values (p<0.05). PTZ alone produced convulsions in 96±4% of the rats. The baseline motor scores were 0.12±0.8.

FIG. 10 shows the comparative effects of KRM-II-82, MP-III-080, and valproate against clonic convulsions induced by pentylenetetrazole (35 mg/kg, s.c.) and on motor performances on an inverted screen in rats. Each point represents the effect in groups of 5 (3 mg/kg dose groups) or 8 (all other groups) rats. Quantal data were analyzed by Fisher's Exact Probability test (*:p<0.05). PTZ, alone produced convulsions in 97±2% of the rats. The baseline motor scores was 0.08±0.1.

FIG. 11 shows the comparative effects of HZ-166, KRM-II-81, KRM-II-82, and diazepam against convulsions induced by pentylenetetrazole (PTZ, i.v.) in rats. Data show dose of PTZ required to induce convulsions as a function of drug dose. Each point represents the mean±SEM effect in groups of 8 rats. Data were analyzed by ANOVA followed by Dunnett's test with * signifying statistically-significant separation from vehicle control values (p<0.05). Each point represents the effects in 8 mice. Baseline values across studies (effects of drug vehicle) was 35.1±1.2

FIG. 12 shows the comparative effects of HZ-166, KRM-II-81, KRM-II-82, and diazepam in rats that were seizure kindled to daily electrical stimulations of the basolateral amygdala. Each point represents the mean±SEM effect in groups of 8 rats. Data were analyzed by ANOVA followed by Dunnett's teat with * signifying statistically-significant separation from vehicle control values (p<0.05). Seizure free scores (seizure severity=0) were 0/8 for HZ-166, 1/8 for KRM-II-82, 2/8 for diazepam, and 7/8 for KRM-II-81. Additional non-parametric analysis was conducted on the seizure severity data with essentially comparable results.

FIG. 13 shows the dampening effects of KRM-II-81 firing rate frequency (Hz) in tissue resected from juveniles with epilepsy. Data were collected for 1 hour under each control conditions (no KRM-II-81, unfilled circles) or in the presence of 30 μM (KRM-II-81) using either pictrotoxin (left panel) or AP-4 (right panel) as a stimulant of neuronal activity.

FIG. 14A shows the compounds tested at 100 μM in HEK-293T cells transiently transfected with full-length cDNAs encoding human (α2), or rat (α1, α3, α5, γ2L, β3) GABA_{A} receptor subunits and the related current responses to GABA from recording in the whole-cell configuration, with cells voltage-clamped at −50 mV. GABA concentrations were EC₃₋₅ for each receptor isoform. Data were analyzed by two-way ANOVA followed by post-hoc Tukey's multiple comparison test. Data for KRM-II-81 are from Lewter et al. (2017) for comparison. Each bar represents the mean+/−SEM of 3-6 experiments. *p<0.05, **P<0.01; ***p<0.001; ****p<0.0001 comparing response at α2 or α 3 vs. other α subunits.

FIG. 14B shows the concentration effect functions for MP-III-080 (n=3-5 independent experiments) in HEK-293T cells transiently transfected with full-length cDNAs encoding human (α2), or rat (α1, α3, α5, γ2L, β3) GABA_{A} receptor subunits. Concentration effect data for KRM-II-81 is shown in Lewter et al. (2017).

FIG. 15, Left Panel: The effects of KRM-II-81 on immobility times in male NIH Swiss mice in the forced-swim test (FST). Each bar represents the mean+/−SEM. The number of animals per treatment group was 7-8, with the exception of imipramine (imi) (15 mg/kg), n=6. Right Panel: The effects of KRM-II-81 on immobility times in C57BL/6 males in the tail-suspension test (TST). Each bar represents the mean+/−SEM. The number of animals per treatment group was 6-8; n=8 for all groups except 30 mg/kg, for which n=6. *p<0.05; ***p<0.0001 compared to vehicle control values (veh) by Dunnett's test.

FIG. 16. Left Panel: The effects of KRM-II-82 on immobility times in male ME Swiss mice in the forced-swim test (FST). Each bar represents the mean+/−SEM. The number of animals per treatment group was 7-8, with the exception of imipramine (IMI) (15 mg/kg), n=6. Right Panel: The effects of MP-III-080 on immobility times in male Swiss mice in the forced-swim test (FST). Each bar represents the mean+/−SEM. The number of animals per treatment group was 8. *p<0.05; **p<0.01 compared to vehicle control values (veh) by Dunnett's test.

FIG. 17. Top Panel: Effects of diazepam and β-CCT alone and in combination on the inverted-screen assay. Each bar represents the mean+/−SEM (n=8 mice/group). *p<0.05; compared to vehicle control values (veh) by Dunnett's test. Bottom Panel: Effects of diazepam and β-CCT alone and in combination in the forced swim test. Each bar represents the mean+/−SEM (n=8 mice/group), *p<0.05; **p<0.01 compared to vehicle control values (veh) by Dunnett's test.

### DETAILED DESCRIPTION

Interest in treating pain in a non-addictive fashion as well as in the absence of tolerance or no development of tolerance by admixing an α1 GABA subtype preferring antagonist with a clinically employed benzodiazepine or their halo, acetylene analogs at the C7 position forms the basis of this discovery. For example, α1 GABA subtype preferring antagonists include 3-PBC, 3-ISOPBC, βCCt, WYS8 and their corresponding salts as well as related analogs (Cook, J. M. et al. U.S. Pat. No. 8,268,854, Sep. 18, 2012).

The clinically employed benzodiazepine doses may be admixed with appropriate dose of the α1 GABA subtype preferring antagonist (for example 1 to 30 mg/kg) to antagonize the effects mediated by α1 benzodiazepine GABAergic subtypes. This may result in antinociceptive agents that do not develop tolerance, as well as are devoid of side effects including sedation, ataxia, amnesia, and addiction. These mixtures may also provide anticonvulsants and anxiolytics, with little or no side effects including tolerance.

α1 GABA subtype preferring antagonists for example are 3-PBC, 3-ISOPBC, βCCt, WYS8 and their corresponding salts as well as related analogs and have been safely employed in rodents (H. June et al. Brain Research, 1998, 794, 103; M. Savić et al. Pharmacol. Biochem. Behav, 2004, 79, 279; M. Savić et al. Psychopharm. 2005, 180, 455; Joksimovic, S. et al. European J. Neuropsychopharmacology, 2013, 23, 390; Divljaković, J. et al. Brain Res. Bull, 2013, 91, 1); squirrel and rhesus monkeys (S. Lelas et al. Psychopharmacology, 2002, 161, 180; D. Platt et al. Psychopharmacology, 2002, 164, 151; J K Rowlett et al. Psychopharmacology, 2003, 165, 209; D. Platt. et al. J Pharm. Exp. Therapeut, 2005, 313, 658; S. Licata et al. Psychopharmacology, 2009, 203, 539) as well as baboons (Kaminski, B. et al. Psychopharmacology, 2013, 227, 127; August, Weerts, Cook et al. Drug and Alcohol Dependence, 2016, 170, 25).

One of the major importances in this invention is the ability to choose a dose that antagonizes only the adverse effects at α1β2/3γ2 GABA_{A} receptors. α1 GABA subtype preferring antagonists can be admixed with any benzodiazepine agonist used in the clinic for anxiety or epilepsy, which may provide a combination treatment that has antinociceptive activity, devoid of tolerance with little or no addiction. α1 GABA subtype preferring antagonists (in contrast to flumazenil which can antagonize all the benzodiazepine-sensitive receptors) may be admixed with the halo and acetyleno analogs of clinically used benzodiazepines as well to provide anxiolytic, anticonvulsant, and antinociceptive effects. Recently a two-step regiospecific synthetic scalable route for preparation of α1 GABA subtype preferring antagonists has been developed (V. V. N. Phani Babu Tiruveedhula, et. al Org Biomol Chem 2015, 13, 10705).

In summary, this invention relates to use of any benzodiazepine anxiolytics and anticonvulsants (and their C (7) halogen or acetyleno analogs) with an α1β2/3γ2 antagonist or α1β2/3γ2 preferring antagonist (for example βCCt, 3-PBCH.Cl, 3-ISOPBC.HCl, WYS8, and related analogs) to treat anxiety, epilepsy, depression, or pain in the absence of tolerance. This combination may antagonize the sedative, ataxic, amnesic and some or all of the addictive properties of clinically employed benzodiazepine agonists. The combination may also be used with any α2 and α3 agonists including but not limited to KRM-II-81, KRM-II-18B, MP-III 080, NTS16085, and related analogs admixed with α1β2/3γ2 antagonists or α1β2/3γ2 preferring antagonists to provide antinociceptive agents with no tolerance as well as anxiolytic and anticonvulsant effects devoid of adverse effects.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein; whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons. Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and ten-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain saturated hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methythexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond. Lower alkenyl contain 2 to 6 carbon atoms.

The term “alkynyl,” as used herein, means a straight or branched, hydrocarbon chain containing at least one carbon-carbon triple bond. Lower alkynyl include 2 to 6 carbon atoms.

The term “alkoxyalkyl,” as used herein, refers to an alkoxy group, as defined herein, appended to the parent molecular moiety through an alkyl group, as defined herein.

The term “alkoxyfluoroalkyl,” as used herein, refers to an alkoxy group, as defined herein, appended to the parent molecular moiety through a fluoroalkyl group, as defined herein.

The term “alkylene,” as used herein, refers to a divalent group derived from a straight or branched saturated chain hydrocarbon, for example, of 1 to 6 carbon atoms. Representative examples of alkylene include, but are not limited to, —CH₂CH₂—, —CH₂CH₂CH₂—, —CH₂CH₂CH₂CH₂—, and —CH₂CH₂CH₂CH₂CH₂—.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkyl group (e.g., indanyl), a phenyl group (i.e., naphthyl), or a non-aromatic heterocycle (e.g., benzo[d][1,3]dioxol-5-yl, 2,3-dihydrobenzo[b][1,4]dioxin-6-yl).

The term “cyanoalkyl,” as used herein, means at least one —CN group, is appended to the parent molecular moiety through an alkylene group, as defined herein.

The term “cyanofluoroalkyl,” as used herein, means at least one —CN group, is appended to the parent molecular moiety through a fluoroalkyl group, as defined herein.

The term “cycloalkoxy,” as used herein, refers to a cycloalkyl group, as defined herein, appended to the parent molecular moiety through an oxygen atom.

The term “cycloalkyl,” as used herein, refers to a carbocyclic ring system containing zero heteroatoms and zero double bonds. Representative examples of cycloalkyl include, but are not limited to, cyclopropyl, cyclobutyl, cyclopentyl, cyclohexyl, cycloheptyl, cyclooctyl, cyclonenyl cyclodecyl, adamantyl, and bicyclo[1.1.1]pentanyl.

The term “cycloalkenyl,” as used herein, means a non-aromatic monocyclic or multicyclic ring system containing at least one carbon-carbon double bond and preferably having from 5-10 carbon atoms per ring. The cycloalkenyl may be monocyclic, bicyclic, bridged, fused, or spirocyclic. Exemplary monocyclic cycloalkenyl rings include cyclopentenyl, cyclohexenyl, cycloheptenyl, and bicyclo[2.2.1]heptenyl.

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “fluoroalkoxy,” as used herein, means at least one fluoroalkyl group, as defined herein, is appended to the parent molecular moiety through an oxygen atom. Representative examples of fluoroalkoxy include, but are not limited to, difluoromethoxy, trifluoromethoxy and 2,2,2-trifluoroethoxy.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl airlines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaryl (bicyclic heteroaryl). The monocyclic heteroaryl are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered six membered aromatic monocyclic rings have three double bonds. The bicyclic heteroaryl is an 8- to 12-membered ring system having a monocyclic heteroaryl ring fused to a monocyclic aromatic, saturated, or partially saturated all-carbon ring, a monocyclic heteroaryl, or a monocyclic heterocycle. The bicyclic heteroaryl is attached to the parent molecular moiety at an aromatic ring atom. Representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, 6,7-dihydro-1,3-benzothiazolyl, imidazo[1,2-a]pyridinyl (e.g., imidazo[1,2-a]pyridin-6-yl), naphthyridinyl, pyridoimidazolyl, thiazolo[5,4-b]pyridin-2-yl, thiazolo[5,4-d]pyrimidin-2-yl.

The term “heterocycle” or “heterocyclic,” as used herein, means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a monocyclic heterocycle fused to a monocyclic heteroaryl, or a Spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocycle is attached to the parent molecular moiety at a non-aromatic ring atom (e.g., 2-oxaspiro[3.3]heptan-6-yl, indolin-1-yl, hexahydrocyclopenta[b]pyrrol-1(2H)-yl). Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentatene, hexahydro-2H-2,5-methanocyclopenta[b]furan, hexahydro-1H-1,4-methanocyctopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocycles are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “hydroxyfluoroalkyl,” as used herein, means at least one —OH group, is appended to the parent molecular moiety through a fluoroalkyl group, as defined herein.

In some instances, the number of carbon atoms in a hydrocarbyl substituent (e.g., alkyl or cycloalkyl) is indicated by the prefix “C_{x-y}”, wherein x is the minimum and y is the maximum number of carbon atoms in the substituent. Thus, for example, “C₁₋₃alkyl” refers to an alkyl substituent containing from 1 to 3 carbon atoms.

The term “sulfonamide,” as used herein, means —S(O)₂NR^{z}— or —NR^{z}S(O)—, wherein R^{z} may be hydrogen, alkyl, cycloalkyl, aryl, heteroaryl, heterocycle, alkenyl, or heteroalkyl.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

The term “allosteric site” as used herein refers to a ligand binding site that is topographically distinct from the orthosteric binding site.

The term “modulator” as used herein refers to a molecular entity (e.g., but not limited to, a ligand and a disclosed compound) that modulates the activity of the target receptor protein.

The term “ligand” as used herein refers to a natural or synthetic molecular entity that is capable of associating or binding to a receptor to form a complex and mediate, prevent or modify a biological effect. Thus, the term “ligand” encompasses allosteric modulators, inhibitors, activators, agonists, antagonists, natural substrates and analogs of natural substrates.

The terms “natural ligand” and “endogenous ligand” as used herein are used interchangeably, and refer to a naturally occurring ligand, found in nature, which binds to a receptor.

In the context of treating a disorder, the term “therapeutically effective amount” as used herein refers to an amount of the compound or a composition comprising the compound which is effective, upon single or multiple dose administrations to a subject, in treating a cell, or curing, alleviating, relieving or improving a symptom of the disorder in a subject. A therapeutically effective amount of the compound or composition may vary according to the application. In the context of treating a disorder, a therapeutically effective amount may depend on factors such as the disease state, age, sex, and weight of the individual, and the ability of the compound to elicit a desired response in the individual. In an example, a therapeutically effective amount of a compound is an amount that produces a statistically significant change in a given parameter as compared to a control, such as in cells (e.g., a culture of cells) or a subject not treated with the compound.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2, METHODS OF USE

In one aspect, the invention provides methods of treating a disorder selected from the group consisting of pain, epilepsy, and depression comprising administering to a subject in need thereof, a therapeutically effective amount of a GABA_{A} agonist, or a pharmaceutically acceptable salt thereof, and a α1β2/3γ2 GABA inhibitor, or a pharmaceutically acceptable salt thereof, in an amount effective to inhibit an adverse effect mediated by the GABA_{A} agonist.

The invention also provides a pharmaceutical combination comprising a GABA_{A} agonist, or a pharmaceutically acceptable salt thereof, and a α1β2/3γ2 GABA inhibitor, or a pharmaceutically acceptable salt thereof, for use in the treatment of a disorder selected from the group consisting of pain, epilepsy, and depression. The invention also provides a pharmaceutical combination comprising a GABA_{A} agonist, or a pharmaceutically acceptable salt thereof, and a α1β2/3γ2 GABA inhibitor, or a pharmaceutically acceptable salt thereof, for use in the treatment of a disorder selected from the group consisting of pain, epilepsy, and depression, wherein the treatment has a reduced GABA_{A} agonist-mediated adverse effect compared to use of a GABA_{A} agonist alone in the treatment of pain, epilepsy, or depression. The invention further provides a pharmaceutical combination comprising a GABA_{A} agonist, or a pharmaceutically acceptable salt thereof, and a α1β2/3γ2 GABA inhibitor, or a pharmaceutically acceptable salt thereof, for use in the treatment of a disorder selected from the group consisting of pain, epilepsy, and depression, wherein the treatment has a greater therapeutic window relative to a GABA_{A} agonist-mediated adverse effect than use of the GABA_{A} agonist alone in the treatment of pain, epilepsy, or depression. The invention further provides a GABA_{A} agonist for use in a method of treating pain, epilepsy, or depression, wherein the method comprises the use/administration of the GABA_{A} agonist and a α1β2/3γ2 GABA inhibitor. The invention further provides a α1β2/3γ2 GABA inhibitor for use in a method of treating pain, epilepsy, or depression, wherein the method comprises the use/administration of a GABA_{A} agonist and the α1β2/3γ2 GABA inhibitor. The use of the pharmaceutical combination comprises use of a therapeutically effective amount of the GABA_{A} agonist, or a pharmaceutically acceptable salt thereof, and an amount of the α1β2/3γ2 GABA inhibitor, or a pharmaceutically acceptable salt thereof, effective to inhibit an adverse effect mediated by the GABA_{A} agonist.

The invention also provides the use of a pharmaceutical combination of a GABA_{A} agonist, or a pharmaceutically acceptable salt thereof, and a α1β2/3γ2 GABA inhibitor, or a pharmaceutically acceptable salt thereof, for the preparation of a medicament for the treatment of pain, epilepsy, or depression. The invention also provides the use of a pharmaceutical combination of a GABA_{A} agonist, or a pharmaceutically acceptable salt thereof, and a α1β2/3γ2 GABA inhibitor, or a pharmaceutically acceptable salt thereof, for the preparation of a medicament for the treatment of pain, epilepsy, or depression, wherein the treatment has a reduced GABA_{A} agonist-mediated adverse effect compared to use of a GABA_{A} agonist alone in the treatment of pain, epilepsy, or depression. The use of the pharmaceutical combination comprises a therapeutically effective amount of the GABA_{A} agonist, or a pharmaceutically acceptable salt thereof, and an amount of the α1β2/3γ2 GABA inhibitor, or a pharmaceutically acceptable salt thereof, effective to inhibit an adverse effect mediated by the GABA_{A} agonist.

In the methods and uses described herein, the GABA_{A} agonist and the α1β2/3γ2 GABA inhibitor (pharmaceutical combination) may be administered/used simultaneously, separately, or sequentially, and in any order, and the components may be administered separately or as a fixed combination. For example, the delay of progression or treatment of diseases according to the invention may comprise administration of the first active ingredient in free or pharmaceutically acceptable salt form and administration of the second active ingredient in free or pharmaceutically acceptable salt form, simultaneously or sequentially in any order, in jointly therapeutically effective amounts or effective amounts, e.g. in daily dosages corresponding to the amounts described herein. The individual active ingredients of the combination can be administered separately at different times during the course of therapy or concurrently in divided or single dosage forms. The instant invention is therefore to be understood as embracing all such regimes of simultaneous or alternating treatment and the term “administering” is to be interpreted accordingly. Thus, a pharmaceutical combination, as used herein, defines either a fixed combination in one dosage unit form or separate dosages forms for the combined administration where the combined administration may be independently at the same time or at different times.

The disclosed methods and combinations relate to treatment of anxiety disorders, depression, epilepsy, schizophrenia, and/or pain. In some embodiments, the disorder is selected from the group consisting of pain, epilepsy, and depression. In further embodiments, the disorder is pain. In still further embodiments, the disorder is inflammatory pain, neuropathic pain, or nociceptive pain. In other embodiments, the disorder is epilepsy. In other embodiments, the disorder is depression.

Anxiety disorder is a term covering several different forms of a type of mental illness of abnormal and pathological fear and anxiety. Current psychiatric diagnostic criteria recognize a wide variety of anxiety disorders. Recent surveys have found that as many as 18% of Americans may be affected by one or more of them. The term anxiety covers four aspects of experiences an individual may have: mental apprehension, physical tension, physical symptoms and dissociative anxiety. Anxiety disorder is divided into generalized anxiety disorder, phobic disorder, and panic disorder; each has its own characteristics and symptoms and they require different treatment. The emotions present in anxiety disorders range from simple nervousness to bouts of terror. Standardized screening clinical questionnaires such as the Taylor Manifest Anxiety Scale or the Zung Self-Rating Anxiety Scale can be used to detect anxiety symptoms, and suggest the need for a formal diagnostic assessment of anxiety disorder.

Particular examples of anxiety disorders include generalized anxiety disorder, panic disorder, phobias such as agoraphobia, social anxiety disorder, obsessive-compulsive disorder, post-traumatic stress disorder, separation anxiety and childhood anxiety disorders.

Depression is a state of low mood and is generally caused by genetic, psychological and social factors. Depression can leave those affected feeling down and unable to enjoy activities. Approximately 4.3% of the world population suffers from depression, while lifetime prevalence ranges from 8-12%. Particular examples of depression are major depressive disorder, persistent depressive disorder and bipolar disorder, which itself has extreme lows as a characteristic.

Epilepsy is a common chronic neurological disorder that is characterized by recurrent unprovoked seizures. These seizures are transient signs and/or symptoms due to abnormal, excessive or synchronous neuronal activity in the brain. There are many different epilepsy, syndromes, each presenting with its own unique combination of seizure type, typical age of onset, EEG findings, treatment, and prognosis. Exemplary epilepsy syndromes include, e.g., Benign centrotemporal lobe epilepsy of childhood, Benign occipital epilepsy of childhood (BOEC), Autosomal dominant nocturnal frontal lobe epilepsy (ADNFLE), Primary reading epilepsy, Childhood absence epilepsy (CEA), Juvenile absence epilepsy, Juvenile myoclonic epilepsy (JME), Symptomatic localization-related epilepsies, Temporal lobe epilepsy (TLE), Frontal lobe epilepsy, Rasmussen's encephalitis, West syndrome, Dravet's syndrome, Progressive myoclonic epilepsies, and Lennox-Gastaut syndrome (LCiS). Genetic, congenital, and developmental conditions are often associated with epilepsy among younger patients. Tumors might be a cause for patients over age 40. Head trauma and central nervous system infections may cause epilepsy at any age.

Schizophrenia is a mental disorder characterized by a breakdown of thought processes and by poor emotional responsiveness. It most commonly manifests itself as auditory hallucinations, paranoid or bizarre delusions, or disorganized speech and thinking, and it is accompanied by significant social or occupational dysfunction. The onset of symptoms typically occurs in young adulthood, with a global lifetime prevalence of about 0.1-0.7%. Diagnosis is based on observed behavior and the patient's reported experiences. Genetics, early environment, neurobiology, and psychological and social processes appear to be important contributory factors. Current research is focused on the role of neurobiology, although no single isolated organic cause has been found. Particular types of schizophrenia include paranoid type, disorganized type, catatonic type, undifferentiated type, residual type, post-schizophrenic depression and simple schizophrenia.

Pain is the most common symptom of disease and the most frequent complaint with which patients present to physicians. Pain is commonly segmented by duration (acute vs. chronic), intensity (mild, moderate, and severe), and type (nociceptive vs. neuropathic). Nociceptive pain is the most well known type of pain, and is caused by tissue injury detected by nociceptors at the site of injury. After the injury, the site becomes a source of ongoing pain and tenderness. This pain and tenderness are considered “acute” nociceptive pain. This pain and tenderness gradually diminish as healing progresses and disappear when healing is complete. Examples of acute nociceptive pain include surgical procedures (post-operative pain) and bone fractures. Even though there may be no permanent nerve damage, “chronic” nociceptive pain results from some conditions when pain extends beyond six months. Examples of chronic nociceptive pain include pain from osteoarthritis, rheumatoid arthritis, and musculoskeletal conditions (e.g., back pain), cancer pain, etc.

Neuropathic pain is defined as “pain initiated or caused by a primary lesion or dysfunction in the nervous system” by the International Association for the Study of Pain. Neuropathic pain is not associated with nociceptive stimulation, although the passage of nerve impulses that is ultimately perceived as pain by the brain is the same in both nociceptive and neuropathic pain. The term neuropathic pain encompasses a wide range of pain syndromes of diverse etiologies. The three most commonly diagnosed pain types of neuropathic nature are diabetic neuropathy, cancer neuropathy, and HIV pain. In addition, neuropathic pain is diagnosed in patients with a wide range of other disorders, including trigeminal neuralgia, post-herpetic neuralgia, traumatic neuralgia, fibromyalgia, phantom limb, as well as a number of other disorders of ill-defined or unknown origin.

GABA_{A} agonists may elicit a number of adverse effects at therapeutic doses/amounts. As used herein, an adverse effect mediated by a GABA_{A} agonist or GABA_{A}/benzodiazepine receptor PAM refers to an adverse effect (non-therapeutic effect) resulting from increased activity of a GABA_{A} receptor protein that decreases neuronal excitability. Without being bound by a particular theory, evidence suggests that common adverse effects of a GABA_{A} agonist or GABA_{A}/benzodiazepine receptor PAM result from increased activity of a α1-containing GABA_{A} receptor protein. The adverse effect may be inhibited or blocked by a α1β2/3γ2 GABA inhibitor/antagonist. Common adverse effects include drowsiness, lethargy, fatigue, sedation, impaired motor coordination, ataxia, amnesia, addiction, or tolerance. The amnesia may be impaired long-term memory, including anterograde amnesia or episodic memory loss, as generally described by Griffin, C. E., et al., “Benzodiazepine Pharmacology and Central Nervous System-Mediated Effects,” The Ochsner Journal (2013) 13, 214-223.

Another aspect of the invention provides a method of inhibiting an adverse effect of a GABA_{A} agonist, the adverse effect being selected from the group consisting of tolerance to antinociception, addiction, and drowsiness, comprising administering to a subject in need thereof, an effective amount of a α1β2/3γ2 GABA inhibitor. In another aspect is provided a α1β2/3γ2 GABA inhibitor, or a pharmaceutically acceptable salt thereof, for use in the inhibition of an adverse effect mediated by a GABA_{A} agonist, the adverse effect being selected from the group consisting of tolerance to antinociception, addiction, and drowsiness. Another aspect of the invention provides the use of a α1β2/3γ2 GABA inhibitor, or a pharmaceutically acceptable salt thereof, for the preparation of a medicament for the inhibition of an adverse effect mediated by a GABA_{A} agonist, the adverse effect being selected from the group consisting of tolerance to antinociception, addiction, and drowsiness. In some embodiments, the α1β2/3γ2 GABA inhibitor is provided in an effective amount to inhibit the adverse effect. In some embodiments, the adverse effect is tolerance to antinociception, and an effective amount is a tolerance-inhibiting amount of the α1β2/3γ2 GABA inhibitor.

A therapeutically effective amount of a GABA_{A} agonist, or a pharmaceutically acceptable salt thereof, is an amount that produces a therapeutic effect to treat a disorder when the GABA_{A} agonist is administered to a subject. A therapeutically effective amount of a GABA_{A} agonist, or a pharmaceutically acceptable salt thereof, has a therapeutic effect in the presence of an effective amount of a α1β2/3γ2 GABA inhibitor, as defined herein. Therapeutically effective amounts of clinically used GABA_{A} agonists are well known in the art. Depending on a variety of factors including the particular agent, the condition being treated, and the individual subject, a therapeutically effective amount of a clinically used agent for a human subject may range from 0.25 mg to 30 mg, and may be dosed three to four times daily, for a total daily dose of from about 4 to 120 mg.

The α1β2/3γ2 GABA inhibitors for use in the invention are agents that inhibit an increase in activity of α1-containing GABA_{A} receptor protein that is mediated by GABA_{A} agonists. In some embodiments, the α1β2/3γ2 GABA inhibitor is a α1β2/3γ2 GABA antagonist, i.e., an agent that competitively inhibits the actions of a GABA_{A}/benzodiazepine receptor PAM at α1-containing GABA_{A} receptor proteins, but exerts substantially no effect on basal GABA activity at α1-containing GABA_{A} receptor proteins.

In further embodiments, the α1β2/3γ2 GABA inhibitor selectively inhibits or antagonizes a α1-containing GABA subtype compared to a α2- and/or α3-containing GABA subtype. Selective inhibition/antagonism of a α1-containing GABA subtype compared to a α2- and/or α3-containing GABA subtype can be determined by the relatively greater inhibition, by the selective α1β2/3γ2 GABA inhibitor/antagonist, of an adverse effect versus a therapeutic effect of a GABA_{A} agonist administered in a subject. A selective α1β2/3γ2 GABA inhibitor/antagonist may be referred to herein as a α1 preferring inhibitor/antagonist. At effective amounts, a selective α1β2/3γ2 GABA inhibitor/antagonist inhibits adverse effects mediated by therapeutically effective amounts of a GABA_{A} agonist, without substantially inhibiting the therapeutic effects of the GABA_{A} agonist. In some embodiments, effective amounts of a selective α1β2/3γ2 GABA inhibitor/antagonist inhibit substantially all of one or more adverse effects mediated by a GABA_{A} agonist, without substantially inhibiting the therapeutic effects of the GABA_{A} agonist. In some embodiments, effective amounts of a selective α1β2/3γ2 GABA inhibitor/antagonist inhibit about 30%, 40%, 50%, 60%, 70%, 80%, 90%, or 100% of one or more adverse effects mediated by a GABA_{A} agonist, without substantially inhibiting the therapeutic effects of the GABA_{A} agonist. Amounts of a selective α1β2/3γ2 GABA inhibitor/antagonist that substantially inhibit a therapeutic effect of a GABA_{A} agonist are not considered effective amounts.

In the case of inhibiting tolerance to antinociception, an effective amount of a α1β2/3γ2 GABA inhibitor/antagonist is a tolerance-inhibiting amount of the α1β2/3γ2 GABA inhibitor/antagonist.

Effective amounts of α1β2/3γ2 GABA inhibitor/antagonist may range from approximately 0.1-50 mg per kilogram body weight of the recipient; alternatively about 0.5-20 mg/kg can be administered. Thus, for administration to a 70 kg person, the dosage range could be about 40 mg to 1.4 g. In some embodiments, the compounds are administered more than once per day (e.g. 2×, 3× or 4× per day). In other embodiments, the compounds are administered once a day. Administration may also be less frequent than once a day, e.g., weekly, bi-weekly, monthly, etc. If desired, the effective daily dose may be divided into multiple doses for the purposes of administration.

The combination treatment of a GABA_{A} agonist and α1β2/3γ2 GABA inhibitor/antagonist, as described herein, may provide a greater therapeutic window compared to use of a GABA_{A} agonist alone. A greater therapeutic window refers to a greater range of therapeutic GABA_{A} agonist dosages or a longer duration of treatment that may be administered before the onset of an adverse effect, or with a reduced incidence of adverse effect, that is mediated by the GABA_{A} agonist. Typically, a greater range of therapeutic dosages allows for administration of a higher dose of GABA_{A} agonist before the onset of an adverse effect, or with a reduced incidence of adverse effect. An increased therapeutic window provides for the achievement and maintenance of therapeutic GABA_{A} agonist plasma levels with reduced incidence of one or more adverse effects compared to use of a GABA_{A} agonist alone. A greater therapeutic window includes a reduction of about 30%, 40%, 50%, 60%, 70%, 80%, 90%, or 100% of one or more adverse effects mediated by a GABA_{A} agonist, at therapeutic GAB AA agonist dosages.

### 3. COMPOUNDS

GABA_{A} agonists for use in the invention are agents that increase the activity of the GABA_{A} receptor protein, thereby decreasing neuronal excitability. In some embodiments, the GABA_{A} agonist is a GABA_{A} positive allosteric modulator (PAM). In some embodiments, the GABA_{A} agonist is a benzodiazepine receptor PAM. The GABA_{A}/benzodiazepine receptor PAM increases the total conduction of chloride ions across the neuronal cell membrane when GABA is bound to its receptor. In some embodiments, the GABA_{A} agonist is an agonist of a benzodiazepine receptor comprising an α2, α3, and/or α5 subunit. In further embodiments, the GABA_{A} agonist is an agonist of a α2β2/3γ2, α3β2/3γ2, and/or α5β2/3γ2 GABA receptor.

GABA_{A}/benzodiazepine receptor PAMs are well known in the art, as described in: Hadjipavlou-Litina D, Hansch C (1994); “Quantitative structure-activity relationships of the benzodiazepines. a review and reevaluation,” Chemical Reviews. 94 (6): 1483-1505; Atack, J. “Development of Subtype-Selective GABAA Receptor Compounds for the Treatment of Anxiety, Sleep Disorders and Epilepsy” GABA and Sleep, Molecular, Functional and Clinical Aspects, Monti, J. M. et al. (Eds.) (2010), Springer Basel AG, pp. 25-72; Clayton, T. et al. Current Medicinal Chemistry (2007) 14, 2755-2775; Atack, J. R. et al., Journal of Psychopharmacology (2010) 25(3), 329-344, which are incorporated herein by reference. In some embodiments, the GABA_{A} agonist is adinazolam, alprazolam, bentazepam, bretazenil, bromazepam, bromazepam, brotizolam, camazepam, chlordiazepoxide, cinazepam, cinolazepam, clobazam, clonazepam, clonazepam, clorazepate, clotiazepam, cloxazolam, delorazepam, deschloroetizolam, diazepam, diclazepam, estazolam, etizolam, flualprazolam, flubromazepam, flubromazolam, fluclotizolam, flunitrazepam, flunitrazepam, flunitrazolam, flurazepam, flutazolam, flutoprazepam, halazepam, ketazolam, loprazolam, lorazepam, lormetazepam, meclonazepam, medazepam, metizolam, mexazolam, midazolam, nifoxipam, nimetazepam, nitemazepam, nitrazepam, nitrazolam, nordiazepam, norflurazepam, oxazepam, phenazepam, pinazepam, prazepam, premazepam, pyrazolam, quazepam, rilmazafone, temazepam, thienalprazolam, tetrazepam, or triazolam. GABA_{A}/benzodiazepine receptor PAMs include active metabolites of clinically used agents such as nordiazepam, chlordiazepoxide, and lorazepam.

In some embodiments, a GABA_{A}/benzodiazepine receptor PAM has selectivity for α2/α3-containing GABA receptors, examples of which include KRM-II-81, KRM-II-82, KRM-II-18B, MP-III-080, and NS16085. Further examples include the compounds described in WO2016/154031 and U.S. Pat. No. 9,597,342, which are incorporated herein by reference. In some embodiments, a GABA_{A}/benzodiazepine receptor PAM is a compound of formula (I),

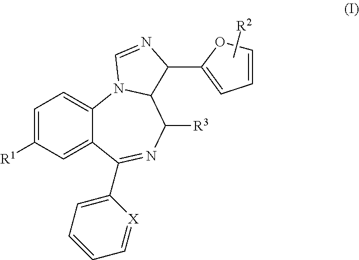

wherein: X is selected from the group consisting of N, C—H, C—F, C—Cl, C—Br, C—I, and C—NO₂; R₁ is selected from the group consisting of —C≡CH, —C≡C—Si(CH₃)₃, -cyclopropyl, bicycle[1.1.1]pentane, and Br; R₂ is selected from the group consisting of —H, —CH₃, —CH₂CH₃ and —CH(CH₃)₂; and R₃ is selected from the group consisting of —H, —CH₃, —CH₂CH₃, —CH(CH₃)₂, —Cl, —CF₃, and —CCl₃. In further embodiments, a GABA_{A}/benzodiazepine receptor PAM is

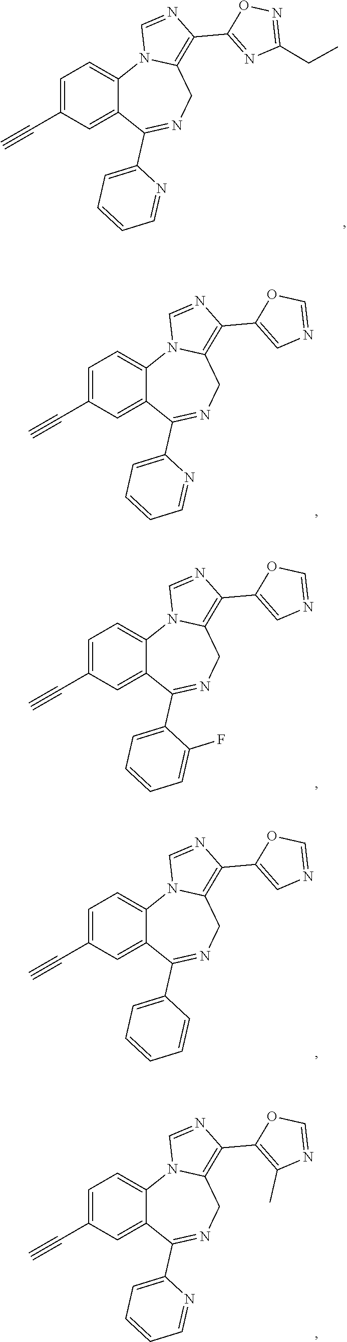

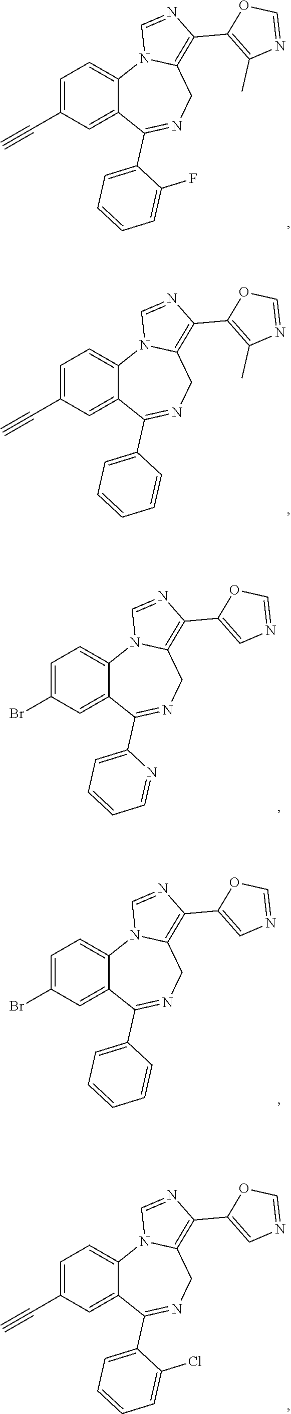 [SMILES: C#Cc1ccc2c(c1)C(c1ccccc1F)=NCc1c(-c3ocnc3C)ncn1-2 ; C#Cc1ccc2c(c1)C(c1ccccc1)=NCc1c(-c3ocnc3C)ncn1-2 ; Brc1ccc2c(c1)C(c1ccccn1)=NCc1c(-c3cnco3)ncn1-2 ; Brc1ccc2c(c1)C(c1ccccc1)=NCc1c(-c3cnco3)ncn1-2 ; C#Cc1ccc2c(c1)C(c1ccccc1Cl)=NCc1c(-c3cnco3)ncn1-2]

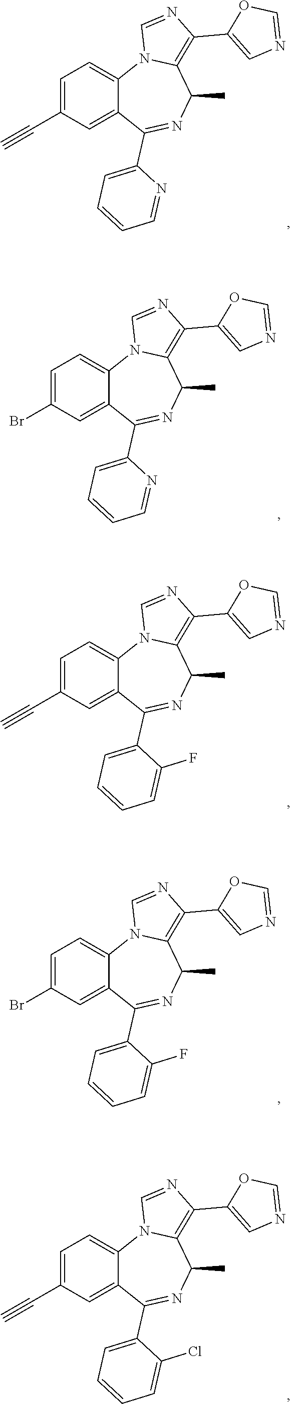 [SMILES: C#Cc1ccc2c(c1)C(c1ccccn1)=N[C@H](C)c1c(-c3cnco3)ncn1-2 ; C[C@H]1N=C(c2ccccn2)c2cc(Br)ccc2-n2cnc(-c3cnco3)c21 ; C#Cc1ccc2c(c1)C(c1ccccc1F)=N[C@H](C)c1c(-c3cnco3)ncn1-2 ; C[C@H]1N=C(c2ccccc2F)c2cc(Br)ccc2-n2cnc(-c3cnco3)c21 ; C#Cc1ccc2c(c1)C(c1ccccc1Cl)=N[C@H](C)c1c(-c3cnco3)ncn1-2]

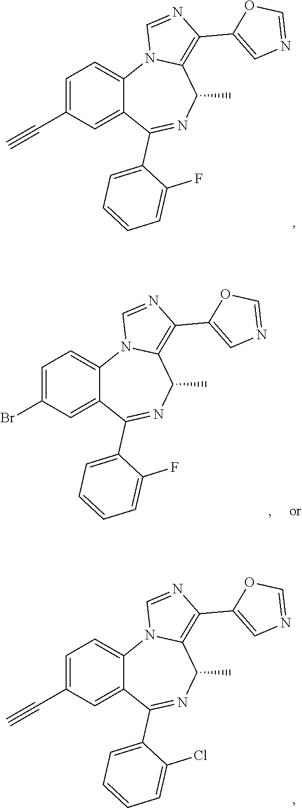 [SMILES: C#Cc1ccc2c(c1)C(c1ccccc1F)=N[C@@H](C)c1c(-c3cnco3)ncn1-2 ; C[C@@H]1N=C(c2ccccc2F)c2cc(Br)ccc2-n2cnc(-c3cnco3)c21 ; C#Cc1ccc2c(c1)C(c1ccccc1Cl)=N[C@@H](C)c1c(-c3cnco3)ncn1-2]

or a pharmaceutically acceptable salt thereof.

In some embodiments, a α1β2/3γ2 GABA inhibitor/antagonist is a compound described in U.S. Pat. No. 8,268,854, which is incorporated herein by reference. Thus, in some embodiments, a α1β2/3γ2 GABA inhibitor is a compound of formula (II),

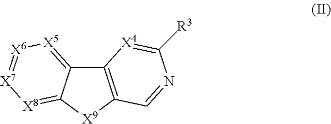

wherein X⁴, X⁵, and X⁸ are each independently N or CH; X⁶ is N, ⁺NR⁶ or CR⁶; X⁷ is N, ⁺NR⁶ or CR⁷; wherein no more than any two of X⁵, X⁶, X⁷ and X⁸ is N; X⁹ is NH, O or S; R³ is CO₂R, OR¹, or COR; R⁶ and R⁷ are independently H, X, aryl, heteroaryl, —C≡CR², lower alkyl, lower alkenyl, or lower alkynyl; R is —C(CH₃)₃₋ₙ(CF₃)ₙ, —C(CH₃)₃₋ᵣ(CH₃₋ₚXₚ)ᵣ, —CH(CH₃)₂₋ₘ(CF₃)ₘ, —CH(CH₃)₂₋ₜ(CH₃₋ₚXₚ)ₜ, aryl, or heteroaryl; R¹ is —CH₂CH₂CH₃, —CH(CH₃)₂, —CH₂CH₂CH₂CH₃, —CH₂CH(CH₃)₂, —CH(CH₃)CH₂CH₃, —CH₂CH₂CH₂CH₂CH₃, —CH₂CH₂CH(CH₃)₂, —CH₂CH(CH₃)CH₂CH₃, or —CH(CH₃)CH₂CH₂CH₃, wherein any of the hydrogens of R¹ is optionally replaced by X; R² is H, lower alkyl, Me₃Si, Et₃Si, n-Pr₃Si, i-Pr₃Si, aryl, or heteroaryl; n is an integer from 0 to 3; m is an integer from 0 to 2; r is an integer from 1 to 3; p is an integer from 1 to 2; t is an integer from 0 to 2; and X is independently F, Cl, Br or I. In further embodiments, a α1β2/3γ2 GABA inhibitor/antagonist is 3-PBC, 3-ISOPBC, 3-CycloPBC, βCCt, or WYS8.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Alogel's Textbook of Practical Organic Chemistry,” 5th edition (1989), Longman Scientific & Technical, Essex CM20 2JE, England, or (2) direct separation of the mixture of optical enantiomers on chiral chromatographic columns, or (3) fractional recrystallization methods.

It should be understood that the compound may possess tautomeric forms, as well as geometric isomers, and that these also constitute embodiments of the disclosure.

The present disclosure also includes an isotopically-labeled compound, which is identical to those recited in formula (I), but for the fact that one or more atoms are replaced by an atom having an atomic mass or mass number different from the atomic mass or mass number usually found in nature: Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e. ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

a. Pharmaceutically Acceptable Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

b. Evaluation of Compounds

Compounds may be analyzed using a number of methods, including receptor binding studies and in vivo methods. For example, the GABA_{A} subunit selectivity of compounds can be evaluated, using competitive binding assays. Compounds can also be evaluated in electrophysiological assays in Xenopus oocytes.

Certain compounds described herein may be GABA_{A} receptor ligands which exhibit anxiolytic activity due to increased agonist efficacy at GABA_{A}/α2, GABA_{A}/α3, GABA_{A}/α_{2/3} and/or GABA_{A}/α5 receptors. The compounds may possess at least 2-fold, suitably at least 5-fold, and advantageously at least a 10-fold, selective efficacy for the GABA_{A}/α2, GABA_{A}/α3, and/or GABA_{A}/α5 receptors relative to the GABA_{A}/α1 receptors. However, compounds which are not selective in terms of their agonist efficacy for the GABA_{A}/α2, GABA_{A}/α3, and/or GABA_{A}/α5 receptors are also encompassed within the scope of the present invention. Such compounds will desirably exhibit functional selectivity by demonstrating anxiolytic activity with decreased sedative-hypnotic/muscle relaxant/ataxic activity due to decreased efficacy at GABA_{A}/α1 receptors.

GABAergic receptor subtype selective compounds which are ligands of the GABA_{A} receptors acting as agonists or partial agonists are referred to hereinafter as “GABA_{A} receptor agonists” or “GABA_{A} receptor partial agonists” or “agonists” or “partial agonists”. In particular, in some embodiments, these are compounds that are ligands of the benzodiazepine (BZ) binding site of the GABA_{A} receptors, and hence acting as BZ site agonists or partial agoniks. Such ligands also include compounds acting at the GABA site or at modulatory sites other than the benzodiazepine site of GABA_{A} receptors.

GABAergic receptor subtype selective compounds act preferably by selectively or preferentially activating as agonists or partial agonists of the GABA_{A}/α2 receptors, GABA_{A}/α3 receptors, or GABA_{A}/α_{2/3} as compared to the GABA_{A}/α₁ receptors, A selective or preferential therapeutic agent has less binding affinity or efficacy to the GABA_{A}/α₁ receptors compared to the GABA_{A}/α₂, GABA_{A}/α₃, or GABA_{A}/α_{2/3} receptors. Alternatively, the agent binds to GABA_{A}/α₁, GABA_{A}/α₂ and GABA_{A}/α₃ receptors with a comparable affinity but exerts preferential efficacy of receptor activation at GABA_{A}/α₂, GABA_{A}/α₃, GABA_{A}/α_{2/3}, or GABA_{A}/α₅ receptors compared to the GABA_{A}/α₁ receptors. A selective agent of the present invention can also have a greater or lesser ability to bind to or to activate GABA_{A}/α₅ receptors relative to GABA_{A}/α₂ and GABA_{A}/α₃ receptors. The Bz/GABA agonists act at the benzodiazepine site of the respective GABA_{A} receptors but are not restricted to this drug binding domain in its receptor interactions.

Other methods for evaluating compounds are known to those skilled in the art. For example, an assessment of anxiolytic effects of compounds can be accomplished objectively and quantitatively with operant-based conflict procedures, as described in Fischer et al. Neuropharmacology 59 (2010) 612-618. Briefly, behavior which is positively reinforced can be suppressed in these procedures by response-contingent administration of a noxious stimulus such as mild electric shock. If a compound has an anxiolytic effect it increases the rates of responding that are normally suppressed by response-contingent delivery of shock. The strength of conflict procedures is their predictive validity with respect to expected therapeutic effects in humans. Results from the Fischer et al. indicate that benzodiazepine-like drugs that have pharmacological activity for α2GABA_{A} and/or α3GABA_{A} receptors and low receptor activity at α 1GABA_{A} and α5GABA_{A} receptors may be useful, particularly as non-sedating anxiolytics and agents to treat neuropathic pain.

Anxiolytic activity and locomotor activity can be evaluated in the light/dark box by a method developed by Crawley (Neurosci Biobehav Rev 1985, 9, 37-44). The light/dark box is an extremely simple noninvasive test for anxiolytic activity. Mice or rats are administered new agents 15-30 minutes prior to testing and placed in the dark portion of the light/dark box. The amount of time it takes the animals to enter the light side and how long they stay versus controls (e.g., diazepam) are a measure of anxiolytic activity. The amount of exploration (or lack thereof) can be used as a preliminary measure of sedation.

The marble burying assay and the elevated plus maze test can also be used to test anxiolytic activity. In the elevated plus maze (Savic et al. Pharmacoi Biochem Behav 2004, 79, 279-290), test compounds can be administrated intraperitoneally 15 minutes prior to testing at which time mice can be placed in the center of the maze under a bright light condition. The number of crosses as well as the time spent in the open and closed arms of the maze for the following 15 minutes can be recorded. Control values for the percentage of entries into the open arms, percentage of time spent in the open arms, and total entries can be correlated to values obtained with controls (e.g., diazepam). Promising compounds may not suppress locomotor activity at up to 100 mg/kg and may be anxiolytic.

For evaluation of potential to treat schizophrenia, compounds may be tested using a mouse model as described in Gill et al. Neuropsychopharmacology 2011, 36: 1903-1911. This mouse model of schizophrenia arises from a development disturbance induced by the administration of a DNA-methylating agent, methylazoxymethanol acetate (MAM), to pregnant dams on gestational day 17. The MAM-treated offspring display structural and behavioral abnormalities, consistent with those observed in human patients with schizophrenia. Antagonism or genetic deletion of the α5GABA_{A} receptor (α5GABA_{A} R) leads to behaviors that resemble some of the behavioral abnormalities seen in schizophrenia, including prepulse inhibition to startle and impaired latent inhibition. The MAM model can be used to show the effectiveness of a benzodiazepine-positive allosteric modulator (PAM) compound selective for the α5 subunit of the GABA_{A}R. In Gill et al., the pathological increase in tonic dopamine transmission in the brain was reversed, and behavioral sensitivity to psychostimulants observed in MAM rats was reduced. The data suggests that such compounds would be effective in alleviating dopamine-mediated psychosis.

Compounds selective for GABA_{A} receptor subunits can be tested for the ability to suppress seizures in several standard rat and mouse models of epilepsy, as described in U.S. Patent Application Publication No. US 2011/0261711. Anticonvulsant activity of compounds can be compared to diazepam. The standard models incorporated into anticonvulsant screening include the maximal electroshock test (MES), the subcutaneous Metrazol test scMet), and evaluations of toxicity (FOX). The data for each condition can be presented as a ratio of either the number of animals protected or toxic (loss of locomotor activity) over the number of animals tested at a given time point and dose.

The MES is a model for generalized tonic-clonic seizures and provides an indication of a compound's ability to prevent seizure spread when all neuronal circuits in the brain are maximally active. These seizures are highly reproducible and are electrophysiologically consistent with human seizures. Subcutaneous injection of the convulsant Metrazol produces clonic seizures in laboratory animals. The scMet test detects the ability of a test compound to raise the seizure threshold of an animal and thus protect it from exhibiting a clonic seizure.

To assess a compound's undesirable side effects (toxicity), animals may be monitored for overt signs of impaired neurological or muscular function. The rotarod procedure in Dunham, M. S. et al. J. Amer. Pharm. Ass. Sci. Ed. 1957, 46, 208-209 is used to test minimal muscular or neurological impairment. Minimal motor deficit is also indicated by ataxia, which is manifested by an abnormal, uncoordinated gait. Animals used for evaluating toxicity are examined before the test drug is administered, since individual animals may have peculiarities in gait, equilibrium, placing response, etc., which might be attributed erroneously to the test substance. In addition to MMI, animals may exhibit a circular or zigzag gait, abnormal body posture and spread of the legs, tremors, hyperactivity, lack of exploratory behavior, somnolence, stupor, catalepsy, loss of placing response and changes in muscle tone.

To further characterize the anticonvulsant activity of compounds, a hippocampus kindling screen can be performed. This screen is a useful adjunct to the traditional MES and scMet tests for identification of a substance potential utility for treating complex partial seizures.

Benzodiazepines can be highly effective drugs in certain treatment paradigms. They are routinely employed for emergency situations such as status epilepticus and other acute conditions, But their use in chronic convulsant diseases has been limited due to side effects such as sedation and with high doses respiratory depression, hypotension and other effects. Further it has long been purported that chronic administration of this class of drugs can lead to tolerance to the anticonvulsant effects. This has limited their utility as first line treatment for chronic anticonvulsant conditions, Discovery of a potent BDZ with a decreased side effect profile and efficacy over extended treatment periods would be highly desirable.

In order to assess the effects of tolerance of compounds, whether tolerance could be detected using a chronic (5 day) dose of the candidate drug can be studied. With typical benzodiazepines (for example diazepam), tolerance to the anticonvulsant effects of the drug are evident before 5 days have passed, consequently studies can be done for only 5 days. The dose to be used may be the predetermined ED50 against the scMet seizure model.

### 4. PHARMACEUTICAL COMPOSITIONS AND FORMULATIONS

In another aspect, the invention provides pharmaceutical compositions comprising one or more compounds of this invention in association with a pharmaceutically acceptable carrier. Such compositions may be in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. It is also envisioned that compounds may be incorporated into transdermal patches designed to deliver the appropriate amount of the drug in a continuous fashion. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture for a compound of the present invention, or a pharmaceutically acceptable salt thereof. When referring to these preformulation compositions as homogeneous, it is meant that the active ingredient is dispersed evenly throughout the composition so that the composition may be easily subdivided into equally effective unit dosage forms such as tablets, pills and capsules. This solid preformulation composition is then subdivided into unit dosage forms of the type described above containing from 0.1 to about 500 mg of the active ingredient of the present invention. Typical unit dosage forms contain from 1 to 100 mg, for example, 1, 2, 5, 10, 25, 50, or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

Suitable dosage level is about 0.01 to 250 mg/kg per day, about 0.05 to 100 mg/kg per day, or about 0.05 to 5 mg/kg per day. The compounds may be administered on a regimen of 1 to 4 times per day, or on a continuous basis via, for example, the use of a transdermal patch.

Pharmaceutical compositions for enteral administration, such as nasal, buccal, rectal or, especially, oral administration, and for parenteral administration, such as intravenous, intramuscular, subcutaneous, peridural, epidural or intrathecal administration, are suitable. The pharmaceutical compositions comprise from approximately 1% to approximately 95% active ingredient or from approximately 20% to approximately 90% active ingredient.

For parenteral administration including intracoronary, intracerebrovascular, or peripheral vascular injection/infusion preference is given to the use of solutions of the subunit selective GABA_{A} receptor agonist, and also suspensions or dispersions, especially isotonic aqueous solutions, dispersions or suspensions which, for example, can be made up shortly before use. The pharmaceutical compositions may be sterilized and/or may comprise excipients, for example preservatives, stabilizers, wetting agents and/or emulsifiers, solubilizers, viscosity-increasing agents, salts for regulating osmotic pressure and/or buffers and are prepared in a manner known per se, for example by means of conventional dissolving and lyophilizing processes.

For oral pharmaceutical preparations suitable carriers are especially fillers, such as sugars, for example lactose, saccharose, mannitol or sorbitol, cellulose preparations and/or calcium phosphates, and also binders, such as starches, cellulose derivatives and/or polyvinylpyrrolidone, and/or, if desired, disintegrators, flow conditioners and lubricants, for example stearic acid or salts thereof and/or polyethylene glycol. Tablet cores can be provided with suitable, optionally enteric, coatings. Dyes or pigments may be added to the tablets or tablet coatings, for example for identification purposes or to indicate different doses of active ingredient. Pharmaceutical compositions for oral administration also include hard capsules consisting of gelatin, and also soft, sealed capsules consisting of gelatin and a plasticizer, such as glycerol or sorbitol. The capsules may contain the active ingredient in the form of granules, or dissolved or suspended in suitable liquid excipients, such as in oils.

Transdermal application is also considered, for example using a transdermal patch, which allows administration over an extended period of time, e.g. from one to twenty days.

### 5. KITS

In one aspect, the disclosure provides kits comprising . . . .

The disclosed kits can be employed in connection with disclosed methods of use.

The kits may further comprise information, instructions, or both that use of the kit may provide treatment for medical conditions in mammals (particularly humans). The information and instructions may be in the form of words, pictures, or both, and the like. In addition or in the alternative, the kit may include the compound, a composition, or both; and information, instructions, or both; regarding methods of application of compound, or of composition, for example with the benefit of treating or preventing medical conditions in mammals (e.g., humans).

The compounds and processes of the invention will be better understood by reference to the following examples, which are intended as an illustration of and not a limitation upon the scope of the invention.

### 6. EXAMPLES

Abbreviations used in the examples that follow are:
EC3: A concentration of GABA eliciting 3% of the maximal GABA-elicited current amplitude of the individual oocyte.
log[M]: Represents the logarithm of molar concentration
β-CCT: Beta-carboline-3-carboxylate-t-butyl ester
KRM-II-81: 5-(8-ethynyl-6-(pyridin-2-yl)-4H-benzo[f]imidazo[1,5-a][1,4]diazepin-3-yl)oxazole
KRM-II-82: 5-(8-ethynyl-6-phenyl-4H-benzo[f]imidazo[1,5-a][1,4]diazepin-3-yl)oxazole
MP-III-080: 3-ethyl-5-(8-ethynyl-6-(pyridin-2-yl)-4H-benzo[f]imidazo[1,5-a][1,4]diazepin-3-yl)-1,2,4-oxadiazole
PAM: positive allosteric modulator
ADD: after discharge duration
ADT: after discharge threshold
AMPA: α-amino-3-hydroxy-5-methyl-4-isoxazolepropionic acid
AP-4: 4-Aminopyridine
CTZ: cyclothiazide
EACSF: excitable artificial cerebral spinal fluid
GYKI 53773 or LY300164: 7-acetyl-5-(4-aminophenyl)-8,9-dihydro-8-methyl-7H-1,3 dioxolo(4,5H)-2,3-benzodiazepine
HZ-166: ethyl 8-ethynyl-6-(pyridin-2-yl)-4H-benzo[f]imidazo[1,5-a][1,4]diazepine-3-carboxylate
LFP: local field potentials
PI: protective index

### Example 1

### Competitive Binding Assays

Assays of Competitive Binding to α1β2/3γ2 GABA_{A} Receptors.

Competition binding assays can be performed in a total volume of 0.5 mL at 4° C. for 1 h using [³H]flunitrazepam as the radioligand (Savić, M. M. Cook, J. M. et al. Progr. Neuro. Psychopharm. Biol. Psy. 2010, 34, 376-386). A total of 6 μg of cloned human GABA_{A} receptor DNA containing desired α subtype along with β2 and γ2 subunits can be used for transfecting HEK 293T cell line using Fugene 6 (Roche Diagnostic) transfecting reagent. Cells are harvested 48 h after transfection, washed with Tris-HCl buffer (pH 7.0) and Tris Acetate buffer (pH 7.4) and resulting pellets stored at −80° C. until assayed. On the day of the assay, pellets containing 20-50 μg of GABA_{A} receptor harvested with hypotonic buffer (50 mM Tris-acetate, pH 7.4, at 4° C.) can be incubated with the radiolabel as previously described. Non-specific binding is defined as radioactivity bound in the presence of 100 μM diazepam and represented less than 20% of total binding. Membranes can be harvested with a Brandel cell harvester followed by three ice-cold washes onto polyethyleneimine-pretreated (0.3%) Whatman GF/C filters. Filters can be dried overnight and then soaked in Ecoscint A liquid scintillation cocktail (National Diagnostics; Atlanta, Ga.). Bound radioactivity is quantified by liquid scintillation counting. Membrane protein concentrations are determined using an assay kit from Bio-Rad (Hercules, Calif.) with bovine serum albumin as the standard.

### Example 2

### FLIPR Assay

The FLIPR functional assay is used to determine the EC₅₀ at the α1 and α3 GABA_{A} receptor subtypes. A high EC₅₀ for the α1 subtype would indicate a low chance of adverse effects, including sedation, ataxia, and muscle relaxation. A low α3 EC₅₀ would indicate potential effectiveness as an anxiolytic, antihyperalgesic, and likely an anticonvulsant. See, for example, Liu et al. (Assay. Drug. Dev. Technol. 2008, 6, 781-6) and Joesch et al, (J. Biomol. Screen. 2008, 13, 218-28).

Compounds tested can be solubilized in DMSO at a 10 mM concentration. GABA is available from Sigma (# A2129) and can be prepared at 100 mM in water. HEK-293 cells are stably transfected with the α1, β3, γ2 GABA A receptor subunits (GenBank accession numbers NM_000806.3, NM_000814.5, and NM_198904.1, respectively) or α3, β3, γ2 (NM_000808 for α3) where obtained from ChanTest Co. (Catalog # CT6216 and C16218, respectively).

Cells are cultivated in Dulbeco's Modified. Eagle's Medium (DMFM, Sigma D5796) supplemented with 10 (?/(Fetal Bovine Serum (FBS, Gibco 16000), 0.5 mg/ml Geneticin (Gibco), 0.04 mg/mL Hygromycin B (Gibco), 0.1 mg/mL Zeocin (Gibco) and 20 mM HEPES (Sigma). Cells are grown at 37° C. in a humidified atmosphere of 5% CO₂. In the experiments described here frozen cells are used. For this purpose, cells are grown and maintained under confluency during 2-3 weeks and then frozen down at different cell densities using Recovery™ Cell Culture Freezing Medium (Gibco). 18 hours prior to the experiment, cells are quickly thawed at 37° C. and seeded on Poly-D-Lys 384 plates (Corning 356663) at a density of 25,000 cells/well and in 25 μL of complete cell medium as described above.

Membrane potential changes induced by the flux of ions through the receptor are measured as relative fluorescence units (RFU) using the Fluorometric Imaging Plate Reader (FLIPR Tetra®, Molecular Devices) and the HIM Membrane Potential Blue Assay kit (Molecular Devices), Prior to the addition of the compounds the medium is removed and cells are loaded with 20 μL of dye prepared in assay buffer composed of Hank's Balanced Salt Solution (HBSS with Ca⁺² and Mg⁺²; Gibco 14025) with 20 mM. Hepes. After 1 hour of incubation at room temperature (RT), the plate is placed into the HIPR instrument and experiments are run adding first 10 μL from the 1^{st} addition plate (compound plate) and after a 3 minute incubation adding 20 μL of the 2^{nd} addition or agonist plate. The response to this last GABA addition is monitored for another 3 minutes.

1^{st} Addition Plates or Compound Plates.

First addition plates containing the compounds to be tested are prepared as follows: compounds in 10 mM dimethyl sulfoxide (DMSO) stock are serially diluted from column 3 to 12 and 13 to 22 in 100% DMSO using Corning 3657 plates and a Tecan Freedom Evo® platform. Then, compounds are further diluted 1:100 in assay buffer. A GABA EC₀ (assay buffer alone) and EC₁₀₀ (150 or 100 μM final GABA concentration after 1^{st} addition for α1 or α3-containing receptor cell lines, respectively) are also included in these plates and used as minimum and maximum response controls, respectively, to analyse any possible compound agonist response.

2^{nd} Addition Plate or Agonist Plate.

Second addition plates are generated using a GABA EC₂₀ to test potentiation profile of the compounds. EC₂₀ and EC₁₀₀ GABA (final assay concentrations) are used as minimum and maximum response controls, respectively. EC₂₀ is 2 or 1.2 μM final GABA concentration for α1 or α3-containing receptor cell lines, respectively.

Data Analysis.

The difference between the maximum and the minimum (Max-Min) fluorescence reached during the first addition or read interval and the second read interval is used for data analysis (agonist and potentiation, respectively). Data was normalized according to the following formula:

$$\% activation = 100 \times ( \frac{Test well - Median EC 0 or 20 Control}{Median EC 100 Control - Median EC 0 or EC 20 Control} )$$

wherein “Test well” refers to those that contain test compounds.

EC₅₀ and maximum stimulation values are determined from concentration-response curves at 10 distinct concentrations. The four-parameter logistic model is used to fit each data set.

### Example 3

### GABA-Evoked Current Responses for Recombinant GABA_{A} Receptors

Electrophysiological recording of Xenoptis laeivs Oocytes.

Xenoptis laeivs frogs were purchased from Xenopus-1 (Dexter, Mich.). Colagenase B was from Boehringer Mannheim (Indianapoli, Ind.). GABA was from RBI. cDNA clones. The rate GABA receptor alpha 1-5, beta 3 and gamma 2 clones were gifts from Dr. Luddnes (Department of Psychiatry, University of Mainz, Germany). Capped cRNA was synthesized from linearized template cDNA encoding the subunits using mMESSAGE mMACHINE kits (Ambion, Austin, Tex.), Oocytes were injected with the alpha, beta, and gamma sununits in a molar ratio of 1:1:1 as determined by UV absorbance. Mature X. laevis frogs were anesthetized by submersion in 0.1% 3-aminobenzoic acid ethyl ester, and oocytes were surgically removed. Follicle cells were removed by treatment with collagenase B for 2 hr. Each oocyte was injected with 50-100 ng of cRNA in 50 ml of water and incubated at 19° C. in modified Barth's saline (88 mM NaCl, 1 mM KCl, 2.4 mM NaHCO₃, 0.41 mM CaCl₂), 0.82 mM MgSO₄, 100 μg/nil gentamicin and 1.5 mM HEPES, pH 7.6). Oocytes were recorded from after 3 to 10 days post-injection.

Electrophysiological Recording.

Oocytes were perfused at room temperature in a Warner Instruments oocyte recording chamber # RC-5/18 (Hamden, Conn.) with perfusion solution (115 mM NaCl, 1.8 mM CaCl₂), 2.5 mM KCl, 10 mM HEPES, pH 7.2). Perfusion solution was gravity fed continuously at a rate of 15 ml/min. Compounds were diluted in perfusion solution, an applied until a peak current was reached.

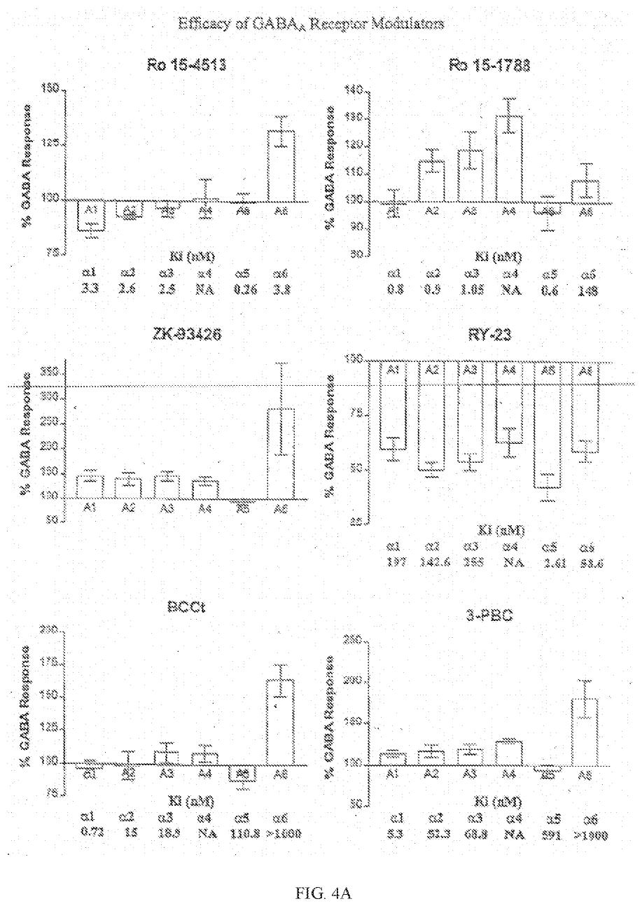

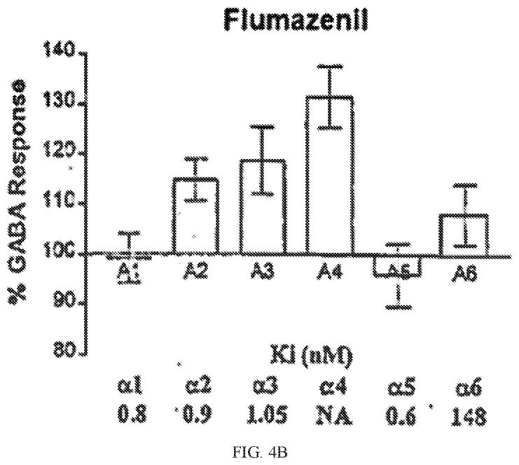

The percent modulation of GABA-evoked current responses in voltage clamped Xenopus oocytes expressing recombinant GABA_{A} receptors was measured (FIG. 4A and FIG. 4B). Each oocyte was injected with cRNA of indicated α subunit together with cRNA of β3 and γ2 subunits. GABA concentration is at the EC50 for each receptor subunit combination. Concentration of indicated modulatory is saturating (1-10 μM). The peak whole cell current response from application of GABA and modulator is reported as the percentage of the peak response to GABA alone (% GABA Response).

### Example 4

### Anxiolytic Activity

Anxiolytic Marble Burying Assay.

The marble burying assay is used to determine the anxiolytic activity of a given compound. Experiments are carried out by the methods described in Li et al, (Life Sciences 2006, 78, 1933-1939). Separate groups of mice are used in these experiments. After 60 min acclimation to the dimly lit experimental room, mice are placed in a 17×28×12 cm high plastic tub with 5 mm sawdust shavings (Harlan Sani-Chips, Harlan-Teklad, Indianapolis, Ind., USA) on the floor, which is covered with 20 blue marbles (1.5 cm diameter) placed in the center. Mice are left in the tub for 30 mM. The number of marbles buried (⅔ covered with sawdust) is counted and submitted to inter-observer reliability assessment. Defensive burying (Broekkamp 1986) is the natural reaction for the mice. When given an anxiolytic, such as diazepam, the mice are less likely to defensively bury the marbles.

Vogel Conflict Model for Anxiety.

The Vogel conflict procedure is used to determine the anxiolytic effects a compound exerts on a test subject, and HZ-166 has previously been shown to be effective in rhesus monkeys.

Experiments are conducted as described in the protocol of Alt et al. (Neuropharmacology 2007, 52, 1482-1487). Experimentally-naive adult male Sprague-Dawley rats (Harlan Industries, Indianapolis, Ind.), weighing between 200 and 300 g, are used as subjects. The rats are housed in Plexiglas cages (4 per cage) and given free access to Lab Diet #5001 for rodents (PMI Nutrition International Inc., St. Louis, Mo.). Water is withheld for 20-24 hours prior to the first training session. A 12-hr light/dark cycle is maintained, and all experimental sessions are conducted during the light phase of the cycle at about the same time each day.

Apparatus.

The experiments are conducted using operant behavior test chambers ENV-007 (Med Associates Inc., Georgia, Vt., USA), 30.5×24.1×29.2 cm. The test chambers are contained within light and sound attenuating shells. On the front wall of the chamber, a food trough is mounted 2 cm off the grid floor on the centerline. Two response levers are centered 8 cm off the centerline and 7 cm off the grid floor. Three lights are located above each response lever at 15 cm off the grid floor. Responding on the levers is without consequences for all sessions. On the rear of the chamber, a sipping tube is mounted 3 cm off the grid floor and 3 cm from the door. The sipping tube is wrapped with electrical tape to prevent the circuit from being completed if the animals are holding/touching the tube. All events are controlled and licking data is recorded by a Compaq computer running MED-PC Version IV (Med Associates Inc., Georgia, Vt., USA).

Sipper Tube Training.

Rats are put into the chamber on day 1 and 2 with white noise and the houselight illuminated, and allowed to drink for a total of six minutes after the first lick is made. The six minutes is broken into two components, the first three minutes is recorded as the unpunished component and the second three minutes are recorded as the punished component. During the two training days no shock is delivered in the punished component. After training, animals are returned to the home cage and given access to water for 30 minutes. For the second and third tests for each group, water is withheld for 24 hours before the training session. Animals are re-trained for one day. After training, animals are returned to the home cage and given access to water for 30 minutes.

Sipper Tube Testing.

On day 3, animals are weighed and injected with either vehicle or compound and returned to the home cage. Thirty minutes after injection, animals are placed into the test chamber. The session is identical to the training session except that during the punishment component the sipper tube delivered a brief electrical shock (100 milliseconds, 0.5 mA) after every 20^{th} lick (FR20). In vehicle punished, is it is expected that the rats hesitate from drinking due to the anxiousness of being shocked. When given an anxiolytic, the mice will continue to drink water despite the electrical shock.

Data Analysis.

The mean number of licks for both the unpunished and punished components are analyzed. In addition, data is also expressed as a percent of control values. The calculation is done using the mean number of licks for the control group in both components. Individual animal means (percent control) are calculated for animals receiving drug utilizing the formula: number of licks divided by mean number of licks by control group times 100 for each respective component. Dose-effect functions are analyzed by ANOVA followed by post-hoc Dunnett's test with vehicle treatment as the control standard. The proportion of animals exhibiting specified numbers of responses is analyzed by Fisher's exact probability test comparing vehicle control to drug values. Statistical probabilities ≤0.05 are considered significant.

### Example 5

### Tactile Hypersensitivity in Spinal Nerve Ligated (SNL) Rats

The von Frey filament test is used to test for antihyperalgesia, or an increased sensitivity to pain. HZ-166 has been shown to perform well in this assay. The von Frey filaments are used to apply pressure to the forelimbs of test subjects at set amounts. When pressure becomes too great, the forelimb is withdrawn and the amount of force applied recorded. The spinal nerve ligation induced hyperalgesia, reducing the amount of force a limb can take before being withdrawn.

Test compounds are given to test the effectiveness of combating the hyperalgesic effect of SNL. Male Sprague-Dawley rats go through SNL at least 90 days prior to the von Frey testing. Rats are first tested without given an injection to determine a baseline. Following baseline establishment, rats (n=5 for all groups) are dosed i.p. with vehicle (1% carboxymethyl cellulose), test compound, or gabapentin (50 mg/kg). Subjects are then tested every hour for four hours to determine the antihyperalgesic effect of the test compounds. For testing, pressure using von Frey filaments is applied to the forelimb of the rat. Pressure is increased until the limb is withdrawn, and the amount of pressure is recorded.

### Example 6

### Complete Freund's Adjuvant Model

Complete Freund's adjuvant (CFA) contains Mycobacterium butyricum, inducing inflammation and an increase in paw thickness. 0.1 mL of CFA was injected in the right hind paw of Sprague Dawley male rats under isoflurane anaesthesia. Mechanical hyperalgesia may be measured 2-3 days after CFA treatment. Rats (n=6) are placed in elevated boxes with a mesh floor. Von Frey filaments (expressed in g) are applied perpendicularly to the hindpaws, starting with the lowest filament (1.4 g) then increased until hindpaw withdrawal is observed. After each measurement, rats receive the next dose of drug (every 20 min) until the maximum threshold (26 g) is observed. For the antagonist study, rats are pretreated with the benzodiazepine site antagonist (10 min) and then receive the next dose of drug (every 20 min) until the pre-CFA threshold was observed.

### Example 7

### α1 Antagonists Reduce the Antihyperalgesic Tolerance to Midazolam

While benzodiazepines are effective and relatively safe for short-term treatment of various neurological disorders, their long-term use is limited due to adverse effects. Previous studies have shown that the sedative properties and physical dependence/addictive properties of benzodiazepines are mostly mediated by GABA_{A} receptors containing α1 subunits. The purpose of the present study was to study the role of α1 GABA_{A} receptors on antihyperalgesic tolerance developed by benzodiazepines. Repeated treatment of α1 antagonists 3-PBC and BCCt were hypothesized to reduce antihyperalgesic tolerance that is developed with long-term treatment of midazolam, when compared with rats that were pretreated with saline.

3PBC.HCl was dissolved in a mixture containing 10% ethanol, 50% propylene glycol, and 40% sterile water. BCCt was dissolved in propylene glycol and then heated in a water bath up to 70° C. Midazolam (Akorn, Inc.) was dissolved in 0.9% saline. Doses were expressed as the weight of the drug in milligrams per kilogram of body weight and drugs were administered intraperitoneally.

Starting 2 days after CFA treatment, mechanical hyperalgesia was measured on days 0, 4, and 8. On days 1-3 and 5-7 rats were pretreated with either 3PBC.HCl (5.6 mg/kg), β-carboline-3-carboxylate-t-butyl ester (BCCt) (3.2 mg/kg) or saline twice a day (a.m. injections (9:00-10:30) and p.m. injections (5:00-6:30)) in their home cages. Rats were treated with midazolam (5.6 mg/kg) immediately after pretreatment of 3PBC.HCl, BCCt, or saline. On days 0 and 4, rats only received p.m. injections due to the mechanical hyperalgesia test done in the a.m.

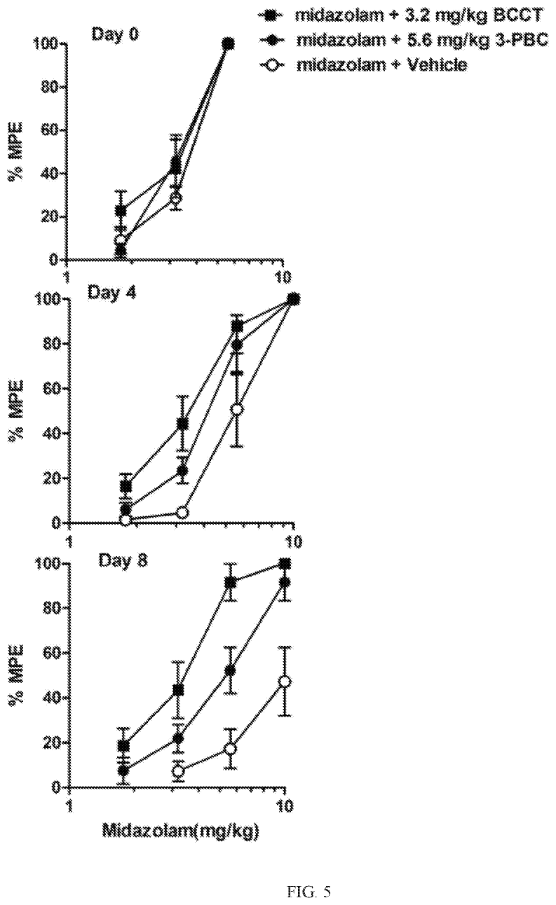

The ability of midazolam to attenuate mechanical hyperalgesia (von Frey assay) was tested (Day 0). Prior to repeated treatment of saline, 3-PBC, or BCCt, midazolam (5.6 mg/kg) resulted in a complete attenuation of mechanical hyperalgesia in all rats (100% maximal possible effect (MPE)). After this test, rats were split into three groups and were repeatedly pretreated with either 3PBC.HCl (5.6 mg/kg), β-carboline-3-carboxylate-t-butyl ester (BCCt) (3.2 mg/kg) or saline twice a day, with a dose of midazolam (5.6 mg/kg) immediately following pretreatment (FIG. 5).

The anti-hyperalgesic effects of midazolam were quantified for each animal as percent maximal possible effect (MPE) for each drug dose by using the following formula: percent MPE=[(post-drug value for a behavioral response (g)−pre-drug value for a behavioral response)/(pre-CFA value-pre-drug value for a behavioral response)×100.

After 7 days of repeated midazolam treatment, rats that were repeatedly treated with saline displayed a great degree of tolerance (as indicated by the rightward shift of the midazolam dose response curve) in that 5.6 mg/kg of midazolam only displayed 17% MPE. Compared to saline-treated animals, rats repeatedly treated with 3-PBC.HCl displayed reduced antihyperalgesic tolerance, where 5.6 mg/kg of midazolam displayed 52% MMPE. Lastly, rats repeatedly treated with BCCt hardly displayed any antihyperalgesic tolerance (no rightward shift of dose response curve) in that 5.6 mg/kg midazolam displayed 92% MPE. Taken together these results suggest that GABA_{A} receptors containing α1 subunits do play a role in the development of antihyperalgesic tolerance in benzodiazepines in that antagonism of these α1 subunits can greatly reduce and prevent the amount of antihyperalgesic tolerance developed by benzodiazepines. In conclusion, data from this study supports the development of selective α1. GABA_{A} antagonists to prevent antihyperalgesic tolerance induced by repeated benzodiazepine treatment.

### Example 8

### Flumazenil Completely Blocked KRM-II-81 Analgesia in a CFA Model

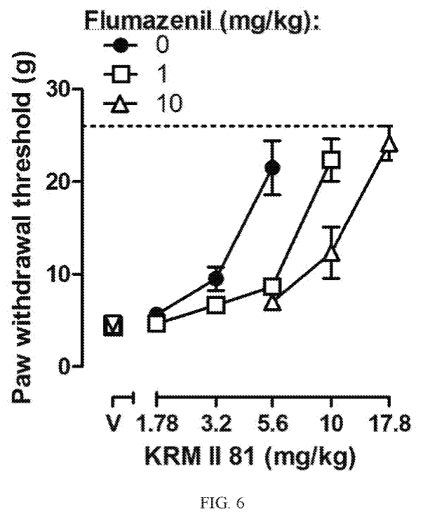

Using a rat model of inflammatory pain (CFA), the mechanism of action of α2/α3-subtype selective GABA_{A} positive allosteric modulator, KRM-II-81, was investigated. KRM-II-81 is an α2/α3 agonist and showed excellent activity against neuropathic pain and anxiety without tolerance, sedation, or ataxia. Rats were pre-treated with the benzodiazepine receptor antagonist flumazenil before receiving cumulative doses of KRM-II-81. Flumazenil dose-dependently attenuated the antinociceptive effects of KRM-III-81 (FIG. 6), confirming that the antinociceptive effects of KRM-II-81 are mediated through the benzodiazepine binding site of GABA_{A} receptors. Flumazenil is different than the α1 preferring antagonists βCCt, 3-PBC, and 3-ISOPBC in that it antagonizes all diazepam-sensitive subtypes.

### Example 9

### Compound Formulation and Administration

Diazepam and β-CCT can be dissolved in 1% hydroxyethylcellulose/0.25% Tween-80/0.05% Dow antifoam in water. KRM-II-81, KRM-II-82, MP-III-080 and HZ-166 can be suspended in carboxymethylcellulose. The dose volume for diazepam and valproate may be 1 ml/kg for rats and 10 ml/kg for mice. HZ-166 and its bioisosteres may be dosed in volumes of 10 ml/kg in mice. HZ-166 may be dosed in a volume of 5 ml/kg in rats and KRM-II-81 may be dosed at 1 ml/kg in rats below doses of 30 mg/kg; 30 mg/kg (dosed at 3 ml/kg), 60 mg/kg (dosed at 6 ml/kg).

Doses and routes of administration of the GABA_{A} compounds were based upon prior in vivo studies with these molecules (Poe et al., J. Med. Chem., 2016 Dec. 8; 59(23): 10800-10806; Witkin et al., Pharmacol Biochem Behav. 2017 June; 157:35-40). Doses tested are shown in the figures and tables. The compounds were generally dosed in ½ log increments from 3 mg/kg to 30 mg/kg with the exception of diazepam that was given at doses beginning at 0.1 mg/kg (PTZ seizure threshold), 0.3 mg/kg (amygdala kindling), and 1 mg/kg (maximal electroshock). HZ-166 was dosed up to 60 mg/kg, and the doses in the 6 Hz model were 10-50 mg/kg. Valproic acid was given at 300 mg/kg. Pentylenetetrazole was given by s.c. injection as a seizure inducer at 35 mg/kg (producing ˜97% of rats to convulse), and was given by i.v. infusion in the studies designed to assess drug effects on seizure thresholds.

### Example 10

### Rat Inverted Screen Test

The motor effects of Diazepam and KRM-II-81 were studied on the inverted screen. The inverted screen test is used to measure whether or not a test compound induces muscle relaxation. When a test subject is placed on a wire screen which is then inverted, the reaction is to climb to the opposite side so they are no longer hanging upside down. If a compound promotes muscle relaxation, the test subjects will either fall off, or hang onto the screen without being able to climb to the opposite side.

Male, Sprague Dawley rats (Harlan Sprague Dawley, Indianapolis, Ind.) were used and weighed 90-110 g when evaluated in experiments. The apparatus consisted of four 13×16 cm squares of round hole, perforated stainless steel mesh (18 holes/square inch, 3/16 inch diameter, ¼ inch staggered centers, 50% open area) mounted 15 cm apart on a metal rod, 35 cm above the table top. Rats were placed onto the top of a wire screen, which was then inverted so that the rats were hanging upside down. Rats were observed for 60 seconds, at which point they were score (0=climbed over; 1=hanging onto screen; 2=fell off).

Male Sprague-Dawley rats (n=5) were dosed i.p. (vehicle=1% carboxymethyl cellulose) with diazepam (3, 10, or 30 mg/kg), KRM-II-81 (10, 30, or 60 mg/kg) or HZ-166 (30 mg/kg) 30 minutes prior to testing. Rats were placed onto the top of a wire screen, which was then inverted so that the rats were hanging upside down. Rats were observed for 60 seconds, at which point they were scored (0=climbed over; 1=hanging onto screen; 2=fell off). Results were analyzed using ANOVA (Dunnett's test: *P<0.05).

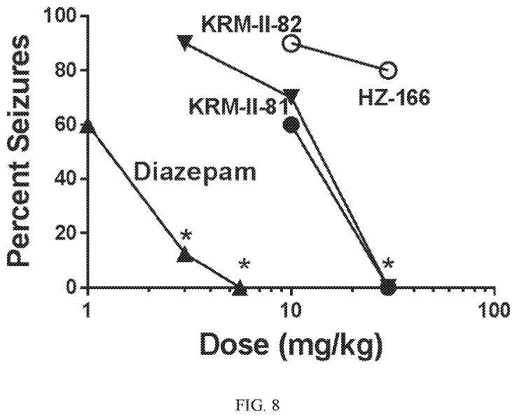

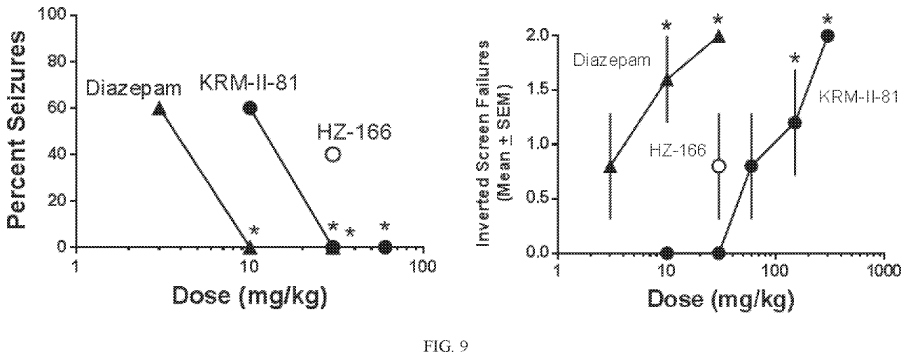

Neither HZ-166 nor KRM-II-81 induced significant muscle relaxation (FIG. 9); however, signs of muscle relaxation began to appear at 30 mg/kg for HZ-166, while the same slight signs occurred at 60 mg/kg for KRM-II-81. Non-dosed rats were able to climb to the top of the screen when inverted (score of 0.4±0.4).

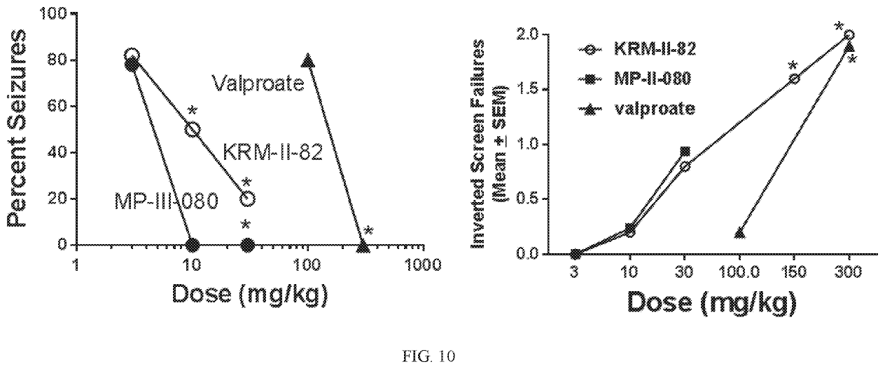

The MTD for producing motor impairment by KRM-II-82 was 150 mg/kg. In contrast, valproate (300 mg/kg), used as a second positive control in addition to diazepam (FIG. 9), produced full motor impairment (FIG. 10).

### Example 11

### Rotarod Assay

The rotorod assay is used to determine the ataxic effects, generally stemming from the α1 subtype, that compounds have in test subjects. Male NIH Swiss mice (n 10/group) are trained on a rotarod (Ugo Basile 7650) at 4 r.p.m. for two minutes per training session prior to testing. On test day, mice are dosed i.p. with either vehicle (1% carboxymethyl cellulose) or one of the test compounds (10 or 30 mg/kg) 30 minutes prior to testing. Once placed on the rotarod, mice are observed for falling. Mice that did not fall off during testing are given a “success” designation, while mice that fell off once during the 2 minutes of testing are scored as “partial.” Mice that fell twice fail the trial.

### Example 1.2

### Maximal Electroshock (MES)-Induced Convulsion Protection

The maximal electroshock (MES) assay is designed to determine how well a test compound can prevent seizures induced by applying a voltage stimuli to a mouse. HZ-166 has previously been shown to be effective in this assay, as well as giving protection against scMET-induced seizures.

Male CD1 mice (n=10) are pretreated i.p. with vehicle or test compound. Mice are subjected to electrical induced tonic seizures and examined for anticonvulsant effects 30 minutes after treatment. Mice are then given a 7 mA electroshock using a Wahlquist Model H for 0.2 seconds and observed for the presence or absence of seizure activity. Each mouse is tested only once and euthanized immediately following the test.

### Example 13

### Mouse Electroshock-Induced Seizure Model

Electroshock-Induced Seizures.

This assay detects effects of compounds that produce generalized seizures and those that dampen seizure spread. Male, CD1 mice (Taconic Farms) were studied at weights of 21-32 g. Mice=10/dose) were used with Wahlquist Model H stimulator with 0.2 sec stimulation with corneal electrodes. Mice were observed for approximately 10 sec after administration of the electrical stimulus (10 uA) and the types of convulsions were recorded (0=no convulsion, 1=clonus, 2=tonic flexion, 3=tonic extension). Mice were euthanized immediately following the test. Tonic extension was used as the primary endpoint. The percentage of animals exhibiting convulsions was analyzed by Fisher's Exact probability test.

Both diazepam, KRM-II-81 and KRM-II-82 fully prevented electroshock-induced seizures in mice with diazepam being 5× more potent than KRM-II-81 to induce full seizure protection. HZ-166 was not efficacious up to 30 mg/kg (FIG. 8, Table 1).

### Example 14

### Rat Pentylenetetrazole-Induced Seizure Model

Pentylenetetrazole (PTZ)-Induced Seizures.

After the inverted screen test, the rats were dosed with pentylenetetrazole (PTZ)(35 mg/kg, s.c.) in a volume of 1 ml/kg and placed in an observation cage (40.6×20.3×15.2 cm) with a floor containing 0.2.5 inches of wood chip bedding material. PTZ dose was determined to be ˜EC₉₇ for producing clonic convulsions. The rats were then observed for 30 min post PTZ for clonus (defined as clonic seizure of fore- and hindlimbs during which the rat demonstrated loss of righting) or for tonic seizures as exemplified by loss of righting accompanied with tonic hindlimb extension. The percentage of animals exhibiting convulsions was analyzed by Fisher's Exact probability test.

Diazepam and KRM-II-81 (Table 1) fully prevented PTZ-induced clonus in rats with diazepam being 10× more potent than KIM-II-81 to induce full seizure protection. HZ-166, while showing a tendency toward efficacy, did not significantly separate from vehicle up to 30 mg/kg (FIG. 9, Table 1). This indicates that KRM-II-81 has greater therapeutic potential against convulsions than HZ-166.

Both KRM-II-82 and MP-III-080 also dose-dependently suppressed convulsions induced by PTZ with MP-II-080 being more potent (FIG. 10). Up to 30 mg/kg, both compounds were without significant effect on motor performance (FIG. 10).

### Example 15

### Pentylenetetrazole (PTZ)-Induced Seizure Threshold

Male F-344 Sprague-Dawley rats (Indianapolis, Ind.) were randomly assigned to treatment groups and dosed with vehicle (vehicle=1% carboxymethyl cellulose) with diazepam (0.1, 0.3, or 1 mg/kg) or test compound (3, 10, 30, or 60 mg/kg) 30 minutes prior to testing. Rats were placed in a restrainer and a winged infusion needle was inserted into the lateral tail vein. Intravenous infusion with 10 mg/ml PTZ at a rate of 0.5 ml/min was initiated until a clonic convulsion occurred, and the time to clonic convulsion was recorded in sec or a maximum of 4 min was recorded. Following infusion, rats are euthanized. The dose of PTZ required to elicit a clonic convulsion was calculated using the infusion rate, concentration of PTZ, time to clonic convulsion, and animal weight. Results were analyzed using ANOVA (Dunnett's test: *P<0.05).

The dose of PTZ required to produce convulsions was 35.1±1.2 mg/kg, which was the dose (35 mg/kg) used to produce clonic convulsions when given as a bolus in the PTZ-induced seizure experiments described above. Pretreatment of rats with diazepam, KRM-II-81, or KRM-II-82 increased the dose of PTZ required to induce convulsions (FIG. 11). Diazepam was about 10 times more potent than the other two molecules but was less efficacious at this dose range (both diazepam and KRM-II-81 were equally efficacious against electroshock and acute PTZ). KRM-II-81 began to exhibit a significant protection against seizures, requiring a 71 mg/kg dose of PTZ when pretreated with 10 mg/kg of KRM-II-81. HZ-166 was not active and displayed little protection up to 60 mg/kg (FIG. 9, Table 1). HZ-166: F_{3,28}=1.1, p=0.36; KRM-II-81: F_{3,28}=8.5, p<0.001; KRM-II-82: F_{2,21}=13.5, p<0.001 (vehicle control values used in ANOVA for this compound); Diazepam: F_{2,21}=1.7, p=0.20.

### Example 16

### Amygdala Kindling

Basolateral Amygdala Kindling.

Seizure kindling models evaluate effects of drugs on the sensitization of the nervous system to seizure induction. In this test, drugs were evaluated for their ability to impact seizure parameters in rats that were fully kindled by daily electrical stimulation of their amygdala. Valproic acid was used as a positive control. Experiments were conducted using the general procedures of Zwart et al. (J. Pharmacol. Exp. Ther., 2014, 351:124-133). A bipolar electrode for electrical stimulation and EEG recording was stereotaxically implanted into one hemisphere of the basolateral amygdala (AP −2.2, ML −4.8, DV −8.5 mm, relative to bregma) of male Wistar rats. After post-operative recovery, electrical kindling begins, where a subthreshold constant current (400 μA, 1 ms, monophasic square-wave pulses, 60 Hz for 1 sec) is given once a day Monday-Friday for about 4-6 weeks until a rat is fully kindled. A fully kindled rat has experienced 10 consecutive stage 5 seizures or 10 of its last 12 were stage 5 according to the Racine Scale.

Twelve fully kindled rats were assigned to this study, and eight rats were selected and randomized to initial compound treatment groups from baseline after discharge threshold (ADT), seizure severity score, and after discharge duration (ADD). A pseudo within-subjects Latin Square design was used for subsequent testing, as replacement rats were used in the event that an assigned rat did not meet the pre-compound testing baseline criteria or a rat lost it head cap during a seizure. On test day, rats were dosed 30 min prior to beginning stimulation. After the pre-treatment, rats were stimulated using an ascending staircase sequence beginning at 10 μA and increasing in log unit steps of 10, 16, 25, 40, 65, 100, 160, 250, and 400 μA. Animals were stimulated until they were assigned a Seizure Severity Score for a visually observably seizure or they reached the 400 μA threshold limit. ADD was determined following testing. ADT was the current that induced a seizure; measurements were scale-adjusted to capture the stimulation scale change required to observe a seizure from the previous baseline to the ADT scored on test day. When seizures were completely blocked, animals were assigned a scaled score of 0 for data analysis. The average scale adjusted ADT was approximately 0.63 μA in vehicle treated rats.

Seizure Severity:

Racine score of behavioral response to stimulation: 0=no behavioral response; 1=immobility, staring and/or facial clonus; 2=head nodding, jaw clonus, and/or tongue protrusion; 3=unilateral forelimb clonus; 4=bilateral forelimb clonus and/or rearing; 5=bilateral forelimb clonus with rearing and loss of balance.

After-Discharge Duration (ADD) is the duration of the first after-discharge.

ADT, ADD, and severity scores were analyzed by ANOVA. Seizure severity data was also analyzed by the nonparametric Skillings-Mack test, a generalization of the Friedman test for one-way repeated measures designs. Posthoc tests when reported were either by Dunnett's method (seizure severity) or using Wilcoxon sign rank tests (ADT and ADD). Valproic acid, used as a positive control was not used in ANOVA calculations.

Data Summarization.

For the in vivo assays, minimal effective doses (MED) were calculated in mg/kg. MED was defined as the lowest dose administered that produced statistically significant protection compared to vehicle control in the anticonvulsant assays. The MED, or minimal toxic dose (MTD) for motor impairment, was the minimal dose required to produce statistically-significant impairment in motor performance. Doses tested are shown in the figures and tables and are generally dosed in ½ log increments. The protective index (PT) was calculated at the MTD/MED. Thus, a PI of 1 indicates that a compound was equipotent in producing motor impairment and producing anticonvulsant effects. A PI >1 indicates that anticonvulsant efficacy was achieved at doses lower than those producing motor impairment.

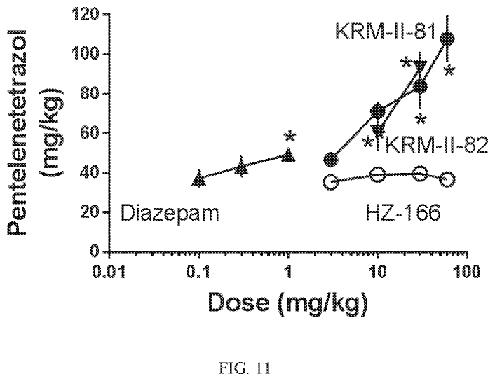

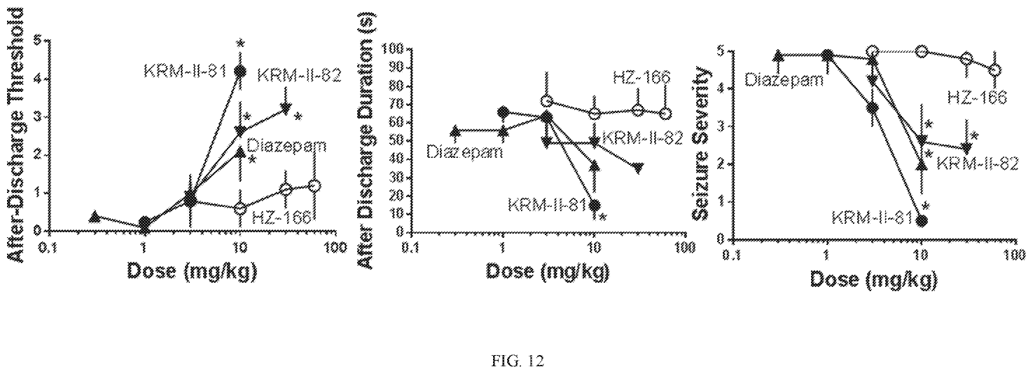

In amygdala kindled rats, diazepam, KRM-II-81, and KRM-II-82 prevented multiple parameters of the seizure induced (FIG. 12). The adjusted after-discharge thresholds (ADT) were significantly increased by diazepam, KRM-II-81 and KRM-II-82, but not by HZ-166. Potencies of diazepam and KRM-II-81 on this dependent measure were approximately equivalent with KRM-II-82 being somewhat less potent (FIG. 12, Table 2), ADT data were scale adjusted to capture the stimulation scale change required to observe a seizure from the previous baseline to the ADT scored on test day. When seizures were completely blocked, animals were assigned a scaled score of 5 for data analysis. The average scale adjusted ADT was 0.75 μA in vehicle treated rats. Since complete block of seizures contributed to the ADT, the seizure free rates are provided. Seizure free scores (seizure severity=0) were 0/8 for HZ-166, 1/8 for KRM-II-82, 2/8 for diazepam, and 7/8 for KRM-II-81.

In contrast, significant decreases in the after-discharge duration (ADD) were produced only by KRM-II-81 with a trend for a significant effect of diazepam and KRM-II-82 at the highest doses tested (FIG. 12, Table 1). The seizure severity score was decreased significantly by diazepam and with a trend toward efficacy in the dose-effect curve for KRM-II-82 but not for HZ-166 itself. Of these molecules, KRM-II-81 was the most efficacious and potent on measures of amygdala kindling.

Statistical analysis confirmed these result descriptions, After-discharge threshold-HZ-166: F_{3,23}=0.9, p=0.92; KRM-II-81: F_{2,21}=27, p<, 0.0001; KRM-II-82: F_{2,21}=3.7, p<0.05; diazepam: F_{3,28}=4.2, p<0.015. After-discharge duration HZ-166: F_{3,28}=0.06, p=0.98; KRM-II-81: F_{2,24}=10.1, p<0.001; KRM-II-82: F_{2,24}=0.76, p=0.48; diazepam: F_{3,28}=1.4, p=0.28. Seizure severity score—HZ-166: F_{4,27}=1.29, p=0.30; KRM-II-81: F_{3,20}=29.21, p<0.0001; KRM-II-82: F_{3,24}=5.28, p<0.01; diazepam; F_{4,27}=10.46, p<0.0001.

A second statistical analysis of the seizure severity data using the Skillings-Macks test confirmed the statistical results presented above. Under analysis by the Skillings-Macks test, the results were as follows for the dose-response analysis: KRM-II-81 (p<0.001), KRM-II-82 (p<0.001), and diazepam (p<0.001), and HZ-166 (p=0.45).

TABLE 1
Comparative potencies of HZ-166, KRM-II-81, and diazepam
across multiple seizure models.

| | | KRM-II- | KRM-II- | MP-III- | |
|---|---|---|---|---|---|
| Assay | HZ-166^{g} | 81 | 82 | 080 | Diazepam^{g} |
| Electroshock^{a} | 30 | 10 | 30 | — | 3 |
| Electroshock to | >30 | 30 | 30 | 5.6 | — |
| Criteria^{b} | | | | | |
| PTZ Clonus | >30 | 30 | 10 | 10 | 10 |
| Potency^{a} | | | | | |
| PTZ Clonus to | >30 | 30 | 30 | 10 | 10 |
| Criteria 1^{b} | | | | | |
| PTZ Threshold | >60 | 10 | 10 | — | 1 |
| Poteny^{a} | | | | | |
| PTZ Threshold | >60 | 30 | 30 | — | >1 |
| to Criteria^{c} | | | | | |
| After-Discharge | >60 | 10 | 10 | — | 10 |
| Threshold | | | | | |
| Potency^{a} | | | | | |
| After-Discharge | >60 | 10 | 30 | — | >10 |
| Threshold | | | | | |
| to Criteria ^{d} | | | | | |
| After-Discharge | >60 | 10 | >30 | — | >10 |
| Duration | | | | | |
| Potency^{a} | | | | | |
| After-Discharge | >60 | 10 | >30 | — | >10 |
| Duration | | | | | |
| to Criteria ^{e} | | | | | |
| Seizure Severity | >60 | 10 | 10 | — | 10 |
| Poteny^{a} | | | | | |
| Seizure Severity | >60 | 10 | >30 | — | 10 |
| to Criteria^{f} | | | | | |
| 6 Hz Potency^{a} | — | 50 | — | — | — |
| 6 Hz Potency^{a} | — | 50 | — | — | — |

^{a}Values are minimal effective doses (MED) in mg/kg. MED was defined as the lowest dose administered that produced statistically significant protection compared to vehicle control. Doses tested are shown in the FIGURES and and tables. The compounds were generally dosed in ½ log increments from 3 mg/kg to 30 mg/kg with the exception of diazepam that was given at doses beginning at 0.1 mg/kg (PTZ seizure threshold), 0.3 mg/kg (amygdala kindling), and 1 mg/kg (maximal electroshock). HZ-166 was dosed up to 60 mg/kg, and the closes in the 6 Hz model were 10-50 mg/kg. Valproic acid was given at 300 mg/kg.
^{b}MED producing maximal protection (0% seizures) in mg/kg
^{c}MED for producing ≥2× PTZ seizure threshold at baseline in mg/kg
^{d}MED for producing after-discharge threshold ≥3 in mg/kg
^{e}MED for producing after-discharge duration ≤30 sec in mg/kg
^{f}MED for producing seizure severity score ≤2
^{g}Bold values in the table represent doses not producing full efficacy comparable to valproic acid 300 mg/kg, i.p) as defined by the efficacy criteria in b, c, d, e and f above
— Data not collected

### Example 17

### 6 Hz-Endured Seizures in Mice

6 Hz Seizure Model.

This seizure model is utilized to screen for drugs that might be detected by other screening approaches. For example, the highly used anticonvulsant leviteracetam was effective in this test but not in the pentylenetetrazole assay. Adult, male mice were subjected to 6 Hz stimulation at 44 mA as originally described by Barton et al. (Epilepsy Res. 2001; 47:217-27) and conducted per protocol of the NTH Anticonvulsant Screening Program (available online from the National Institute of Neurological Disorders and Stroke—6 Hz 44 mA Psychomotor Seizure Model, Mouse). Briefly, a mouse was dosed with KRM-II-81, p.o., and 2 hr later given 6 Hz stimulation for 3 sec delivered through corneal electrodes at 44 mA and observed for the presence or absence of seizure activity. A separate group of mice was dosed with higher doses of KRM-II-81 and observed for potential deficits in motor performance and overt signs of toxicity at 4 hr post dosing.

Mice (n=8) subjected to 6 Hz stimulation were protected by orally-administered KRM-II-81 when tested 2 hr post oral dosing (Table 2). Mild tremor was observed at 100 mg/kg and loss of righting was observed in one 1/8 mice at 150 mg/kg and 1/8 mice at 200 mg/kg, a dose at which more severe tremor was noted in 1/8 mice.

TABLE 2
Effects of orally-administered KRM-II-81 in the 6 Hz seizure model
and observed motor effects.

| Dose | Number protected/number tested | |
|---|---|---|
| 10 | 0/8 | |
| 25 | 3/8 | |
| 50 | 7/8 | |
| Dose | Number with observed motor effects | Observation |
| 50 | 0/8 | |
| 100 | 3/8 | tremors |
| 150 | 5/8 | tremor, unable to grasp |
| 200 | 8/8 | more severe tremor' |
| | | loss of righting |

Mice were tested at 2 hr post dosing for seizures post 6 Hz stimulation and another group was evaluated at 4 hr post dosing for motor side effects.

### Example 18

### Protective Indices

When comparing potencies of diazepam and KRM-II 81 on a measure of motor deficit (inverted screen) to its anticonvulsant potencies (Table 3), a protective index can be calculated as Potency_{inverted screen}/Potency_{anticonvutsant}. PI values >1 indicate a margin between efficacy and side-effect doses; PI values=1 indicate that side-effects and efficacy do not quantitatively separate and a PI value of <1 indicates that the potency to produce efficacy is less than the potency to produce motor impairment. PI values for the various seizure models are shown in Table 3.

TABLE 3
Protective indices (PI) for diazepam, KRM-II-81, KRM-II-82 and
MP-III-080^{a}

| Assay | KRM-II-81 | KRM-II-82 | MP-III-080 | Diazepam |
|---|---|---|---|---|
| PTZ Clonus | 5 | 15 | >3 | 1 |
| PTZ Threshold | 15 | 15 | — | 10 |
| After-Discharge | 15 | 15 | — | 1 |
| Threshold | | | | |
| After-Discharge | 15 | <5 | — | <1 |
| Duration | | | | |
| Seizure Severity | 15 | 15 | — | 1 |
| 6 Hz | 4 | — | — | — |

^{a}PI values values were calculated as the minimal effective dose producing motor impairment/minimal effective doses producing efficacy. Electroshock values were not computed since motor impairment at higher doses was not evaluated. Values for HZ-166 could not be calculated for any measure due to lack of efficacy. Values of ≤1 are highlighted in bold. Values >x could not be assigned a definitive value as the highest dose tested did not significantly impair motor performance.

In order to better estimate the separation between motor side-effects and anticonvulsant efficacy, plasma and brain levels of KRM-II-81 at the ED50 for efficacy (17.3 mg/kg) and the ED50 for motor impairment (121 mg/kg) were determined. (Table 4). Using these unbound drug concentrations, the PI based on drug levels in plasma was 3 and that based upon brain drug levels was 3.8.

TABLE 4
Unbound Plasma and brain concentrations of KRM-II-81 (nM)
after i.p. dosing in male, Sprague-Dawley rats (n = 3).

| Dose (mg/kg, i.p.) | | |
|---|---|---|
| 17.3 | Plasma | 3290 ± 107 |
| | Brain | 1640 ± 141 |
| 121 | Plasma | 10000 ± 2147 |
| | Brain | 6250 ± 1652 |
| 121/17.3 Ratio | Plasma | 3.04 |
| | Brain | 3.81 |

Values are means ± S.E.M. of 3 rats in nM.

### Example 19

### Anticonvulsant Activity and Motor-Impairing Effects of Diazepam and α1 Preferring Antagonist Combination

Diazepam (30 mg/kg, p.o.) markedly and significantly impaired motor performance on the inverted-screen test (2.0+/0 vs 0+/−0 for vehicle, p<0.05, n=5 rats/group). In the presence of the α1-preferring antagonist of GABA_{A} receptor, β-CCT (10 mg/kg, i.p.), the motor-impairing effects of diazepam were completely prevented (0+/−0, p<0.05, n=5 rats/group) but the anticonvulsant effects were retained (PTZ alone=5/5 convulsions: diazepam+PTZ=0/5 convulsions; β-CCT+diazepam+PTZ=0/5 convulsions).

### Example 20

### Electrophysiological Effects in Neuronal Cultures

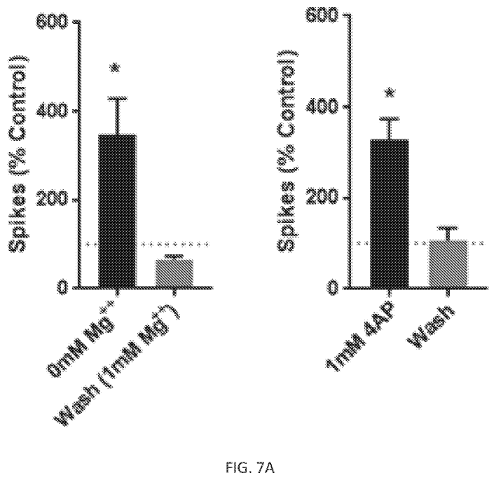

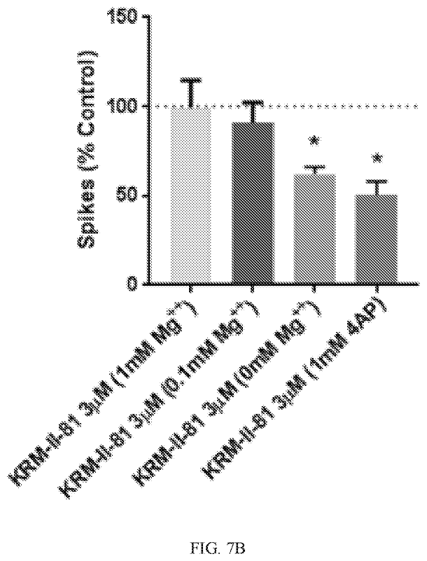

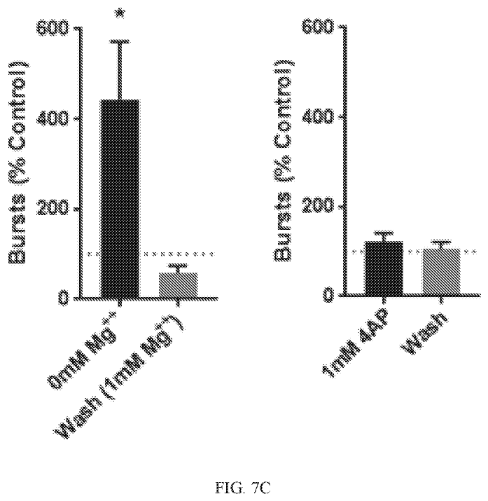

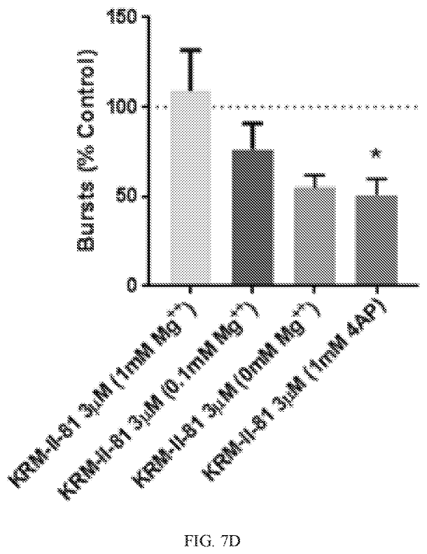

Cultured rat cortical neurons were used to assess the activity of KRM-II-81 on the electrical activity of neurons under basal conditions and under two different conditions of hyper-excitation (FIGS. 7A & 7C). These experiments were conducted to determine if the compounds under investigation were effective in damping aspects of cortical neuronal network activity under basal and hyper-excyted conditions.

Microelectrode Array (MEA) Preparation.

Meastro 12 well plates (Axion Biosystems, Atlanta, Ga.) were treated with a solution of 0.1% polyethylenimine for 2-4 hours, rinsed with sterile H₂O and let dry overnight. Prior to plating of neurons, the MEAs were treated with solution of 20 μg/ml laminin for a minimum of 1 hour.

Primary Neuronal Culture.

Cortices isolated from EIS rats (MEA) were obtained from BrainBits LLC, (Springfield, Ill.) and digested enzymatically with TrypLE Express (Gibco), After 15 minutes of digestion, the tissue was mechanically dissociated with a series of sterile fire-polished glass pipettes of decreasing diameter. The dissociated neurons were plated directly onto substrate-integrated MEA plates and incubated in Nb Active1 (Brainbits LLC, Springfield, Ill.) supplemented with 5% dialyzed fetal bovine serum, 0.25% Glutamax (Gibco). Cell cultures were maintained in tissue incubator (37° C., 6% CO₂) and fed twice a week by exchanging half of the medium. The experiments were performed on DIV 19-25 thus allowing partial maturation of the neurons. Prior to the experiment, the cell culture media was replaced with external buffer containing: 129 mM NaCl, 5 mM KCl, 2 mM CaCl, 1 mM Nigel, 10 mM HEPES, 10 mM glucose. To achieve hyper-excitation the external buffer was modified by removal of magnesium or by addition of 1 mM 4aminopyridine (4-AP).

Mea Recording.

The recordings were obtained at 37° C. using Meastro System with integrated AxIS 2.3 analysis software (Axion Biosystems, Atlanta, Ga.). Channels were sampled simultaneously with a gain of 1200× and a sampling rate of 12.5 kHz/channel. On-line spike detection was done with the AXIS adaptive spike detector. For recordings, a Butterworth band-pass filter (with a high-pass cutoff of 75 Hz and low-pass cutoff of 4000 Hz) was applied along with a variable threshold spike detector set at 7× standard deviation of the rms-noise on each channel. Only wells that did show spontaneous activity (more than 0.3 Hz) on the day planned for the experiment were treated with a compound. Burst was defined as a minimum of 5 spikes occurring with an interspike interval of less than 100 ms. A minimum of one burst per minute was required for bursting analysis. Compounds were added by manual pipetting and the activity was sampled for six minutes prior and post compound addition.

Statistical Analysis.

The data were normalized to baseline activity and reported as mean±standard error of the mean (SEM). For single treatment a single sample t-test was used. To compare between group effects, analysis of variance (ANOVA) with Dunnett's multiple comparison test were utilized; P<0.05 was considered significant.

There was a reversible potentiation of spontaneous neuronal activity by removal of magnesium reflected in increased spiking and bursting frequency. Addition of 1 mM 4-aminopyridine to the external solution primarily increased the frequency of spikes with smaller effect on frequency of bursts.

KRM-II-81 suppressed the hyper-excitation in the network of cortical neurons but not the spontaneous neuronal activity in normal magnesium containing external solution (FIG. 7B, FIG. 7D). In the presence of a normal magnesium containing external solution (1 mM Mg⁺⁺), the addition of 3 μM KRM-II-81 produced no significant change in the frequency of spiking or bursting in neuronal network. When magnesium was omitted from the external solution, 3 μM KRM-II-81 significantly depressed both the frequency of spiking and the frequency of bursting. A similar depression of neuronal activity was observed in the presence of 1 mM 4AP with significant decreases in frequency of spiking and bursting. A smaller, nonsignificant depression of neuronal activity occurred under conditions of reduced magnesium (0.1 mM Mg⁺⁺).

### Example 21

### Human Epileptic Cerebral Cortex Electrophysiology

Patient Data.

Experiments were conducted with tissue from two patients undergoing cortical transection for pharmacologically-refractory epilepsy. The first patient was an 11-year-old female with a history of medically refractory epilepsy with increasing seizure frequency. She presented with localization related (focal), (partial) epilepsy and epileptic syndromes with complex partial seizures and intractable epilepsy. Medications and vagal stimulation failed to prove medically viable. Multiple diagnostics led to the decision to operate. A right frontotemporal parietal craniotomy for resection of her right frontal tumor and seizure focus, using intraoperative Stealth, stereotactic-guided electrocortical graphic localization of her seizure activity, and phase reversal mapping for localization of the central sulcus/precentral gyrus (motor cortex) as well as resection of the anterolateral aspect of the temporal lobe was performed. The anterior 4.5 cm of the middle temporal gyrus, inferior temporal gyrus, fusiform gyrus, and parahippocampal gyrus were resected along with the anterior 3 cm of the superior temporal gyrus on the right side. The hippocampus was left intact.

The second patient was a 12-yr-old boy with medically refractory epilepsy. He underwent a left frontotemporal craniotomy for resection of his anterolateral and mesial left temporal lobe seizure focus using intraoperative electrocorticography and Stealth stereotactic guidance. During surgery, there were abnormal epileptic spikes coming from the electrodes one through three on the left superior temporal gyrus, one through five on the left middle temporal gyrus, left inferior temporal gyrus, and left fusiform gyrus. The anterior 3 cm of the superior temporal gyrus was resected and used for the electrophysiological recordings. The anterior 5 cm of the middle temporal gyrus, inferior temporal gyrus, fusiform gyrus, and parahippocampal gyrus was resected on the left as well. In addition, a left amygdalohippocampectomy was performed without incidence: These later tissues were not studied in the electrophysiological experiments.

Tissue Preparation and Recording.

The tissue was prepared and treated as previously described (Zwart et al., J. Pharmacal. Exp. Ther., 2014, 351:124-133). Slices were maintained at 37° C. and were perfused at 1.0 ml/min first with normal ACSF (NACSF) solution for one hour to see if the tissue was spontaneously active. If spontaneous activity did not develop after one hr, the tissue was bathed in excitable ACSF (EACSF) solution containing 5 mM K⁺ and 0 mM Mg²⁺ to induce robust local field potential (LFP) activity in cortical brain slices. Tissue was then recorded in EACSF with a concentration of 10 μM picrotoxin for 1 hr. Subsequently, tissue was recorded in EACSF with a concentration of 10 μM of picrotoxin with 30 μM KRM-II-81 and recorded for 1 hour. Following the KRM-II-81 addition, tissue was recorded in a washout solution of EACSF and 10 μM Picrotoxin for 1 hr. The concentration of KRM-II-81 was based upon data collected in human cortical slices with perampanel and upon the need to be conservative in concentration estimates to maximize the opportunity to see an effect given limited human tissue opportunities. A second experiment was conducted using AP-4 (50 μM) as an activator instead of picrotoxin.

Recordings were performed on microelectrode arrays of 60 electrodes as previously described (Zwart et al., J Pharmacol. Exp. Ther., 2014, 351:124-133). LFPs with sharp negative peaks below a threshold set at 3 standard deviations of the signal were marked, and the time of the maximum excursion was recorded as the time of that LFP. Time points were binned at 4 ms. Two-way ANOVA was performed to ascertain whether KRM-II-81 was effective and whether the drug effect was dependent upon electrode location.

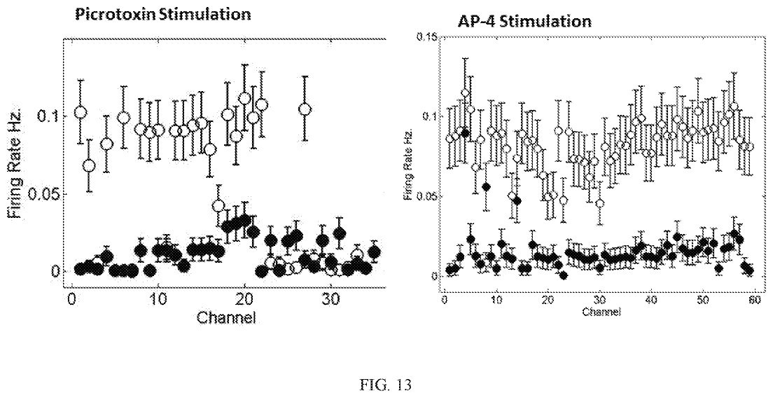

Slices of resected human cortical tissue are typically not active in NACSF alone (Hobbs et al. 2010) as was the case in the current study. By bathing the slice in 10 μM picrotoxin, elevated K and reduced Mg²⁺, however, field potentials were evoked on at least 30 channels of the array. Activity of the slice was first recorded for 1 hr in 10 μM of picrotoxin to establish a baseline. The average firing rate (FIG. 13) was 0.05±0.01 Hz, KRM-II-81 (30 μM) was then added and activity was recorded for another hour again in the presence of 10 μM of picrotoxin. The average firing rate decreased to 0.01±0.005 Hz (FIG. 13). These decreases were statistically significant by two-way ANOVA (F_{1,32}=30.7, p<0.0001), whereas the electrode location was not a significant factor (F_{32,32}=1.1, p=0.36).

Given the exceptional anticonvulsant profile of KRM-II-81, the effect of KIM-II-81 on the firing frequencies in slices from a second epileptic patient was also evaluated. In this second study, firing was stimulated with AP-4. In this slice preparation, activity was observed across all 60 channels of the micro-electrode array. When 30 μM KRM-II-81 was added to the bath, significant attenuation of firing was observed (0.08±0.01 vs. 0.01±0.005) (F_{1,58}=593, p<0.0001), The electrode location was not a significant factor in this drug effect (F_{58,58}=1.2, P=0.22). For both experiments with human tissue, the recovery of firing was evaluated after suppression by KRM-II-81. In both cases, significant recovery was not observed as previously reported with other anticonvulsant mechanisms (Zwart et al., 2014).

Under conditions that have been reported to show the anticonvulsant activity of the AMPA receptor antagonist perampanel, KRM-II-81 also decreased picrotoxin-induced increases in cortical firing rates across an electrode array. In cortical tissue from a second epileptic patient, 4-aminopyridine or AP-4 was utilized as the excitant. KRM-II-81 was also efficacious in suppressing AP-4-enhanced firing (K⁺ channel-driven) in the present study. Thus, the ability or KRM-II-81 to act as an anticonvulsant in human epileptic tissue occurs through general anticonvulsant mechanisms and not due to competition with picrotoxin at GABA_{A} receptors per se. In these experiments, recovery of firing rates post washout of KRM-II-81 was not observed. Importantly, it has been observed from other experiments, that the lack of recovery is not due to run down in the slice, that is, electrical activity is enduring in slices without drug addition. The fact that KRM-II-81 suppressed firing under both picrotoxin stimulation and under AP-4 stimulation increases the generality and strength of these data.

### Example 22

### GABA_{A} Selectivity of KRM-II-81, KRM-II-82, and MP-III-080

Electrophysiological Recordings from Transfected HEK-293T Cells.

Human fibroblast cells (HEK-293T) were transiently transfected with full-length cDNAs encoding human (α2), or rat (α1, α3, α5, γ2L, β3) GABA_{A} receptor subunits using calcium phosphate precipitation. Positively transfected cells were isolated by utilizing co-transfection of a plasmid encoding a surface antibody (pHook™-1, Invitrogen). 20-52 hours following transfection, cells were mixed with magnetic beads coated with antigen specific for the pHook antibody, isolated with a magnetic stand, and then plated onto coverslips and used for patch-clamp recordings 18-28 hours later.

Current responses to GABA were recorded in the whole-cell configuration, with cells voltage-clamped at −50 mV. The bath solution contained (in mM): 142 NaCl, 8.1 KCl, 6 MgCl₂, 1 CaCl₂), and 10 HEPES (4-(2-hydroxyethyl)-1-piperazineethanesulfonic acid) with pH=7.4 and osmolarity adjusted to 295-305 mOsm. The electrode (internal) solution consisted of (in mM); 153 KCl, 1 MgCl₂, 5 K-EGTA (ethylene glycol-bis (β-aminoethyl ether N,N,N′N′-tetraacetate)), and 10 HEPES with pH=7.4 and osmolarity adjusted to 295-305 mOsm. Drug-containing solutions were applied to cells for 5 sec using a computer-driven applicator (open tip exchange <50 msec, SF-77B, Warner Instruments). Currents were recorded with an Axon 200B (Foster City, Calif.) patch clamp amplifier. GABA concentrations were EC₃₋₅ for each receptor isoform, based on previously published data (Picton and Fisher, 2007). Data were analyzed by two-way ANOVA followed by post-hoc Tukey's multiple comparison test. Concentration effect curves for MP-III-080 were generated this assay system as described for KRM-II-81 in Leger et al. (2017).

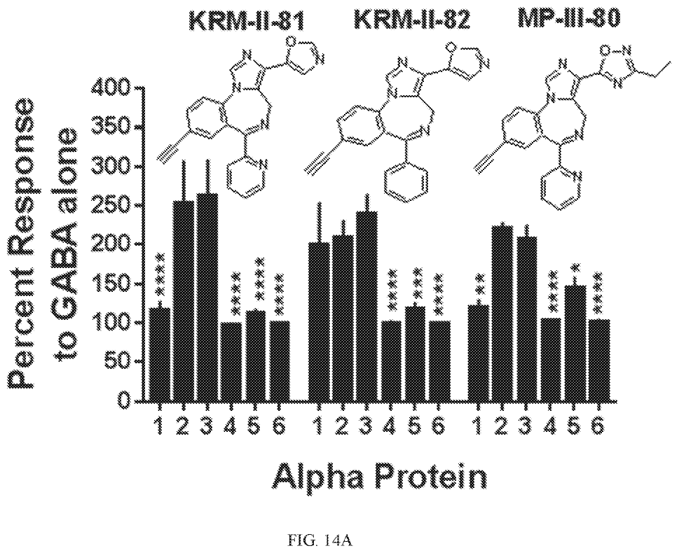

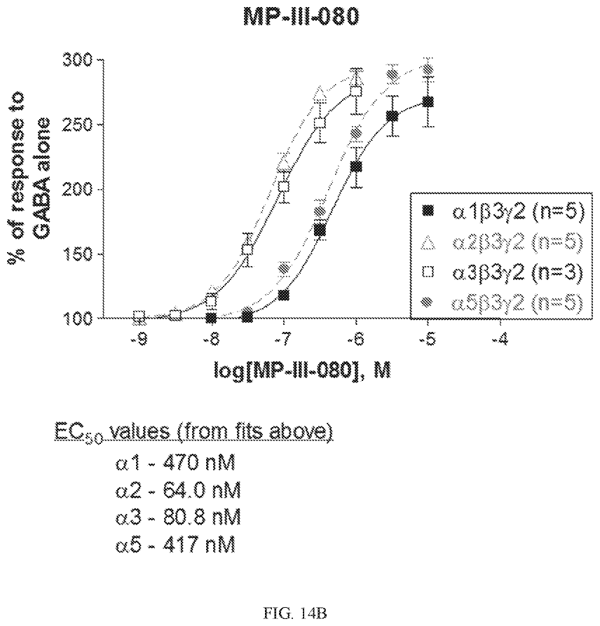

KRM-II-81, KRM-II-82, and MP-III-080 were evaluated for their selectivity for GABA_{A} receptor-associated alpha proteins by testing in HEK-293T cells with a concentration of GABA set at EC₃₋₅ and drug concentrations at 100 nM (FIG. 14A and FIG. 14B). There was a significant difference across alpha subunits (F_{5,50}=63.1, p<0.0001) and a significant drug x subunit interaction (F_{10,50}32 3.81, p<0.001) but not a significant difference across drugs (F_{2,50}=0.94, p=0.40).

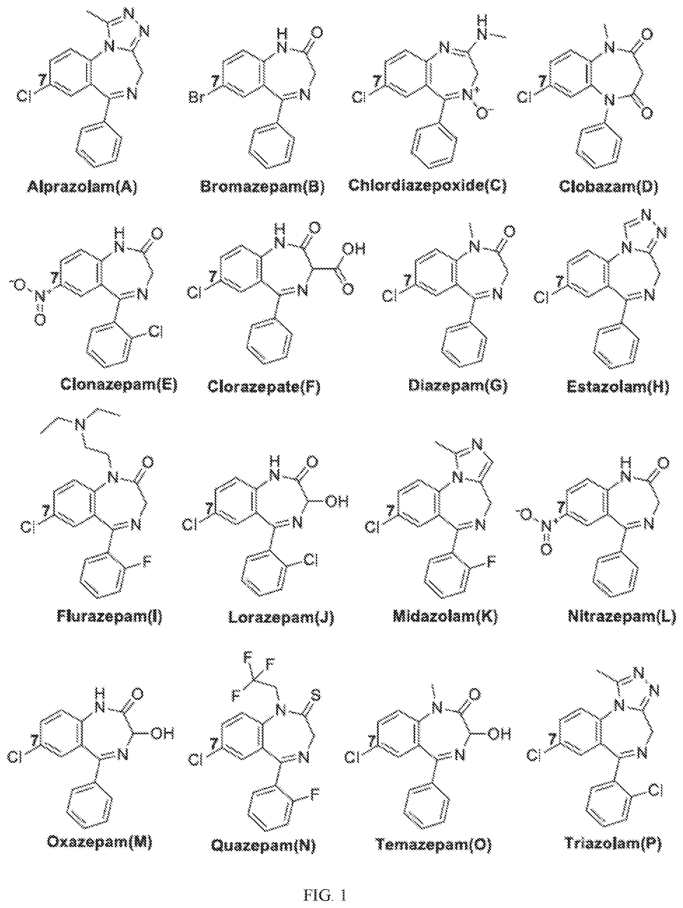

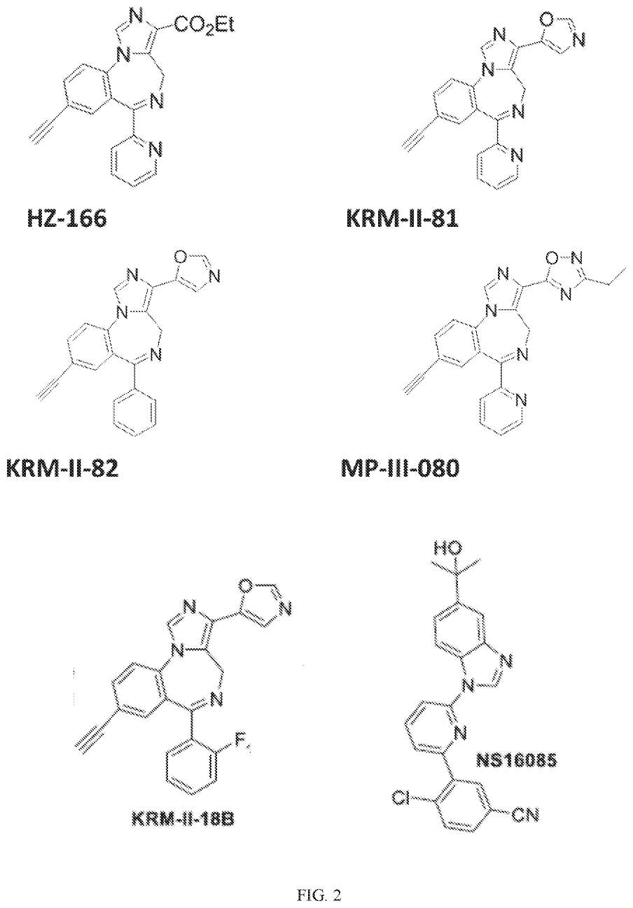

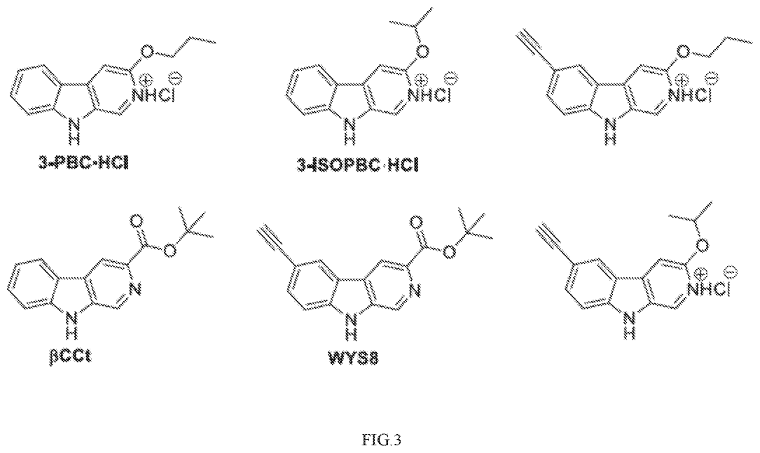

Both KRM-II-81 and MP-III-80 were selective for α2 and α 3 over α1 as reported with other methods (Poe et al., 2016) or the same assay system for KRM-II-81 (Lewter et al., 2017) whereas KRM-II-82 was not (FIG. 14A and FIG. 14B). All three compounds showed a preference for α2 and α 3 over α4, α5 and α6 (FIG. 14A and FIG. 14B). Concentration effect functions for MP-III-080 are shown in the left panel of FIG. 1. These curves substantiate the claims made by the single concentration data in the right panel of FIG. 1 and as established previously for KRM-II-81 (Lewter et al., ACS Chemical Neuroscience, 2017 8(6):1305-1312).

### Example 23

### Antidepressant Activity Model

It has been observed that GABA_{A} receptor PAMs generally do not produce antidepressant-like effects in the forced-swim assay. However, there have been reports of efficacy at some doses in the forced-swim test in some studies with alprazolam, midazolam, and neuroactive steroids. Effects in antidepressant-detecting models are dose-dependent with higher doses generally increasing rather than decreasing immobility times. The antidepressant-associated behavioral effects of a compound can be screened for by using a forced-swim assay which is capable of detecting conventional and novel antidepressants, Mice that are more mobile after a dosing of a compound are determined to be less depressed. The forced-swim assay and tail-suspension assay were used to investigate antidepressant-associated behavioral effects of the GABA_{A} receptor PAMs with selectivity for α2/3 receptors.

Mouse Inverted-Screen Test.

The inverted-screen assay was conducted as previously described (Neuropharmacology, 2017, 126:257-270). Without prior pretraining, mice were placed on an 11 cm×14 cm square of round hole, perforated stainless steel mesh (18 holes/square inch, 3/16 inch diameter, ¼ inch staggered centers, 50% open area) mounted on a metal rod, 35 cm above the table top. The screen was slowly inverted such that the mouse was on the underside of the screen. Mice were then scored for 60 sec as follows: 0=climbed to top of screen, 1=hung onto bottom of screen, 2=fell off. Data were analyzed by ANOVA followed by post-hoc Dunnett's test.

Forced-Swim Assay.

Male NIH Swiss mice (Envigo, Indianapolis, Ind.) were studied. Mice were placed in clear plastic cylinders (diameter: 10 cm; height: 25 cm) filled with 6 cm of water (22-25° C.) for six min. These parameters were minor modifications of the method utilizing 6 cm water depth that has been used on multiple occasions by (e.g, Li et al., Cell Mol Neurobiol 23: 419-430, J Pharmacol Exp Ther 319: 254-259) to detect antidepressant agents of diverse structure and mechanism including tricyclics, monoamine oxidase inhibitors, atypical agents, electroconvusive shock, PDE4 inhibitors, and NMDA receptor antagonists. The duration of immobility during the last four min of the six min test period was scored. A mouse was recorded as immobile when floating motionless or making only those movements necessary to keep its head above water. Data were analyzed by ANOVA followed by post-hoc Dunnett's test.

Tail-Suspension Assay.

Male, C57Bl/6 mice (Envigo, Indianapolis, Ind.) were employed in these experiments. Med Associates Inc. (Fairfax, Vt., USA), apparatus SOF-821 was utilized. The TST was an automated version of published methods in which the tail was secured to a lever on the ceiling of the chamber. The duration of immobility was recorded by a force transducer for a period of 10 min. Data were analyzed by ANOVA followed by post-hoc Dunnett's test.

Compounds.

The test compounds were suspended in 1% hydroxyethylcellulose/0.05% Tween 80/0.05% Dow antifoam. Imipramine was dissolved in water. Compounds were administered in a volume of 10 ml/kg and were given 30 min prior to testing via the i.p. route of administration; diazepam was dosed orally, 30 min prior to testing.

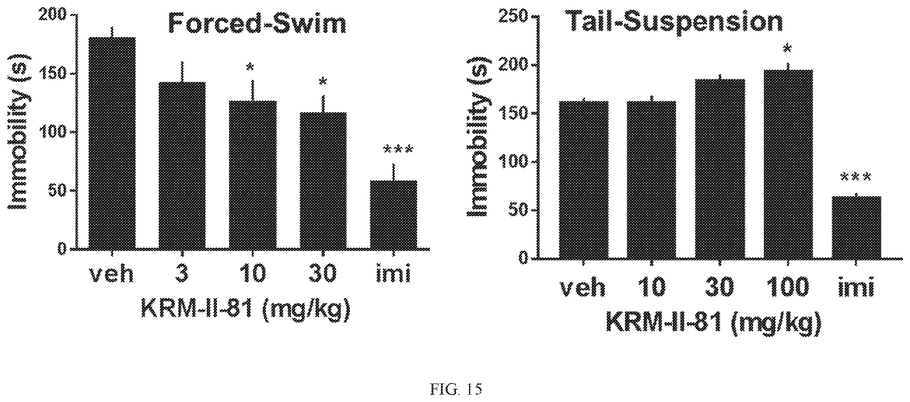

KRM-II-81 produced a moderate, dose-dependent decrease in immobility in NIH Swiss mice in the forced-swim assay (FIG. 15, Left) with imipramine (15 mg/kg) producing a more pronounced reduction in immobility (F_{4,32}=8.19, p<0.0001). In the tail-suspension test in C57Bl/6 mice (FIG. 15, Right), KRM-II-81 produced a slight increase in immobility at the higher doses tested with 100 mg/kg producing effects significantly greater than vehicle control values (F_{4,33}=51.8, p<0.0001). Mice at these doses also exhibited moderate sedation-like effects.

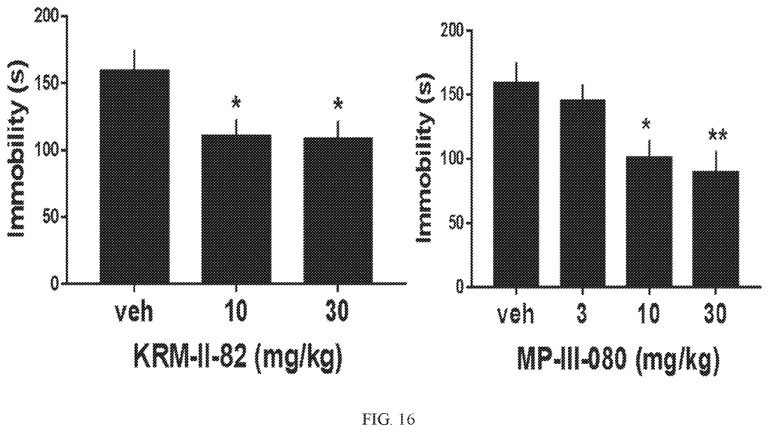

KRM-II-82 also significantly decreased immobility time in NIH Swiss mice (F_{2,21}=4.15, p<0.05) (FIG. 16, Left) as did MP-III-080 (F_{3,28}=5.11, p, 0.01) (FIG. 16, Right).

All three of the structurally-related [1,5-α][1,4]diazepines studied here produced an antidepressant-related behavioral signature in mice. This includes KRM-II-82, which unlike KRM-II-81 and MP-III-080, did not produce anxiolytic-like efficacy in the Vogel conflict test in rats, raising the possibility that anxiolytic and antidepressant effects of this mechanism can be chemically dissociated. Since KRM-II-82 is also not selective for α2/3 vs. α1 protein, as are the other two molecules. KRM-II-81 was not active in the tail-suspension test.

### Example 24

### Antidepressant Activity of Diazepam and α1 Preferring Antagonist Combination

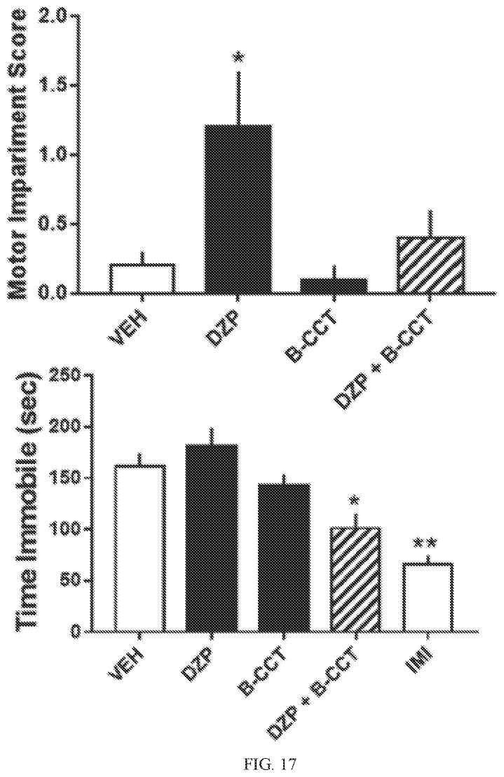

Diazepam was studied at 22 mg/kg, p.o. in the mouse swim test. This dose is based upon prior data in which 30 mg/kg, p.o. produced nearly full motor impairment scores (1.8 out of 2.0) on the inverted screen test. At 22 mg/kg, the motor impairment was still significant but not maximal (F_{3,28}=4.53, p<0.05) (FIG. 17, Top). In the presence of β-CCT (10 mg/kg, i.p., dose based upon prior data), there was a trend for motor-impairing effect of diazepam to be attenuated (p=0.09) and there was no significant motor-impairing effect of the drug combination.

In the forced-swim assay, diazepam did not produce an antidepressant-like effect. In contrast, in the presence of β-CCT, diazepam significantly decreased immobility times like that of the tricyclic antidepressant imipramine (F_{4,33}=10.1, p<0.0001) (FIG. 17, Bottom). The effect of diazepam alone and that produced by diazepam+β-CCT (p<0.01 by Tukey's multiple comparison test).

That the motor-impairing effects of diazepam might be responsible for its lack of efficacy in the forced-swim assay was evaluated in the present study. At doses of the α1-preferring antagonist of GABA_{A} receptors, β-CCT, that block the motor effects of benzodiazepine receptor agonists, the motor-impairing effects of diazepam showed a trend toward attenuation (inverted-screen test). Under these dosing conditions, the effect of diazepam was transformed from inactivity (no decrease in immobility) in the forced-swim test into an antidepressant-like effect. However, higher doses of diazepam anticipated to increase immobility times were not tested. This was the first time that the contribution of α1 receptors to the effects of benzodiazepine agonists in the forced-swim assay has been identified. The data confirm that motor-impairment (measured in the inverted-screen test) significantly contribute to the negative effects of these ligands in antidepressant drug screens (forced-swim). Since the attenuation of the α1 component of diazepam leaves the α2/3 component intact and creates an antidepressant-like behavioral phenotype in mice, the data further implicated α2/3-containing GABA_{A} receptors as drivers of antidepressant-like efficacy.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A pharmaceutical combination comprising a GABA_{A} agonist, or a pharmaceutically acceptable salt thereof, and a α1β2/3γ2 GABA inhibitor, or a pharmaceutically acceptable salt thereof, for use in the treatment of a disorder selected from the group consisting of pain, epilepsy, and depression.

2. The pharmaceutical combination for use of claim 1, wherein the treatment has a reduced GABA_{A} agonist-mediated adverse effect compared to use of the GABA_{A} agonist alone in the treatment of pain, epilepsy, or depression.

3. The pharmaceutical combination for use of claim 1, wherein the treatment has a greater therapeutic window relative to a GABA_{A} agonist-mediated adverse effect than use of the GABA_{A} agonist alone in the treatment of pain, epilepsy, or depression.

4. The pharmaceutical combination for use of claim 2 or 3, wherein the adverse effect is selected from the group consisting of tolerance to a therapeutic effect of the GABA_{A} agonist, addiction, drowsiness, ataxia, sedation, and amnesia.

5. The pharmaceutical combination for use of any of claims 1-4, wherein the use is simultaneous, separate, or sequential.

6. The pharmaceutical combination for use of any of claims 1-5, wherein the disorder is pain.

7. The pharmaceutical combination for use of any of claims 1-6, wherein the disorder is inflammatory pain, neuropathic pain, or nociceptive pain.

8. The pharmaceutical combination for use of any of claims 1-5, wherein the disorder is epilepsy.

9. The pharmaceutical combination for use of any of claims 1-5, wherein the disorder is depression.

10. The pharmaceutical combination for use of any of claims 1-9, wherein the GABA_{A} agonist is a benzodiazepine receptor positive allosteric modulator.

11. The pharmaceutical combination for use of any of claims 1-9, wherein the GABA_{A} agonist is an agonist of a benzodiazepine receptor comprising an α2, α3, or α5 subunit.

12. The pharmaceutical combination for use of any of claims 1-9, wherein the GABA_{A} agonist is an agonist of a α2β2/3γ2, α3β2/3γ2, and/or α5β2/3γ2 GABA receptor.

13. The pharmaceutical combination for use of any of claims 1-9, wherein the GABA_{A} agonist is adinazolam, alprazolam, bentazepam, bretazenil, bromazepam, bromazolam, brotizolam, camazepam, chlordiazepoxide, cinazepam, cinolazepam, clobazam, clonazepam, clonazepam, clorazepate, clotiazepam, cloxazolam, delorazepam, deschloroetizolam, diazepam, diclazepam, estazolam, etizolam, flualprazolam, flubromazepam, flubromazolam, fluclotizolam, flunitrazepam, flunitrazepam, flunitrazepam, flurazepam, flutazolam, flutoprazepam, halazepam, ketazolam, loprazolam, lorazepam, lormetazepam, meclonazepam, medazepam, metizolam, mexazolam, midazolam, nifoxipam, nimetazepam, nitemazepam, nitrazepam, nitrazepam, nordiazepam, norflurazepam, oxazepam, phenazepam, pinazepam, prazepam, premazepam, pyrazolam, quazepam, rilmazafone, temazepam, thienalprazolam, tetrazepam, or triazolam.

14. The pharmaceutical combination for use of any of claims 1-9, wherein the GABA_{A} agonist is MP-III-080 or a compound of formula (1) 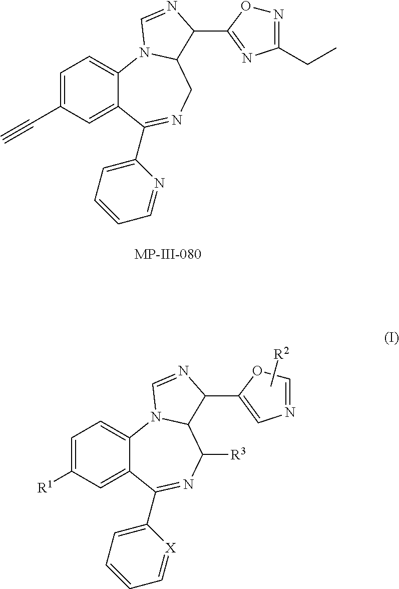
wherein:
X is selected from the group consisting of N, C—H, C—F, C—Cl, C—Br, C—I, and C—NO₂;
R₁ is selected from the group consisting of —C≡CH, —C≡C—Si(CH₃)₃, -cyclopropyl, bicycle[1.1.1]pentane, and Br;
R₂ is selected from the group consisting of —H, —CH₃, —CH₂CH₃ and —CH(CH₃)₂; and
R₃ is selected from the group consisting of —H, —CH₃, —CH₂CH₃, —CH(CH₃)₂, —F, —Cl, —CF₃, and —CCl₃.

15. The pharmaceutical combination for use of any of claims 1-14, wherein the α1β2/3γ2 GABA inhibitor is a α1β2/3γ2 GABA antagonist.

16. The pharmaceutical combination for use of any of claims 1-15, wherein the α1β2/3γ2 GABA inhibitor is a selective inhibitor of a α1-containing GABA subtype compared to a α2- and/or α3-containing GABA subtype.

17. The pharmaceutical combination for use of any of claims 1-4, wherein the α1β2/3γ2 GABA inhibitor is a compound of formula (II), 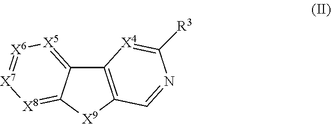
wherein
X⁴, X⁵, and X⁸ are each independently N or CH;
X⁶ is N, ⁺NR⁶ or CR⁶;
X⁷ is N, ⁺NR⁶ or CR⁷;
wherein no more than any two of X⁵, X⁶, X⁷ and X⁸ is N;
X⁹ is NH, O or S;
R³ is CO₂R, OR¹, or COR;
R⁶ and R⁷ are independently H, X, aryl, heteroaryl, —C≡CR², lower alkyl, lower alkenyl, or lower alkynyl;
R is —C(CH₃)₃₋ₙ(CF₃)ₙ, —C(CH₃)₃₋ᵣ(CH₃₋ₚXₚ)ᵣ, —CH(CH₃)₂₋ₘ(CF₃)ₘ, —CH(CH₃)₂₋ₜ(CH₃₋ₚXₚ)ₜ, aryl, or heteroaryl;
R¹ is —CH₂CH₂CH₃, —CH(CH₃)₂, —CH₂CH₂CH₂CH₃, —CH₂CH(CH₃)₂, —CH(CH₃)CH₂CH₃, —CH₂CH₂CH₂CH₂CH₃, —CH₂CH₂CH(CH₃)₂, —CH₂CH(CH₃)CH₂CH₃, or —CH(CH₃)CH₂CH₂CH₃, wherein any of the hydrogens of R′ is optionally replaced by X;
R² is H, lower alkyl, Me₃Si, Et₃Si, n-Pr₃Si, i-Pr₃Si, aryl, or heteroaryl;
n is an integer from 0 to 3;
M is an integer from 0 to 2;
r is an integer from 1 to 3;
p is an integer from 1 to 2;
t is an integer from 0 to 2; and
X is independently F, Cl, Br or I.

18. The pharmaceutical combination for use of any of claims 1-14, wherein the α1β2/3γ2 GABA inhibitor is 3-PBC, 3-ISOPBC, 3-CycloPBC, βCCt, or WYS8.

19. A α1β2/3γ2 GABA inhibitor, or a pharmaceutically acceptable salt thereof, for use in the inhibition of an adverse effect mediated by a GABA_{A} agonist, the adverse effect being selected from the group consisting of tolerance to antinociception, addiction, and drowsiness.

20. A α1β2/3γ2 GABA inhibitor, or a pharmaceutically acceptable salt thereof, for use in the inhibition of tolerance to an antinociceptive effect of a GABA_{A} agonist.

21. The α1β2/3γ2 GABA inhibitor for use of claim 19 or 20, wherein the GABA_{A} agonist is the GABA_{A} agonist of any of claims 10-14.

22. The α1β2/3γ2 GABA inhibitor for use of any of claims 19-21, wherein the α1β2/3γ2 GABA inhibitor is the α1β2/3γ2 GABA inhibitor of any of claims 15-18.

23. A method of treating a disorder selected from the group consisting of pain, epilepsy, and depression comprising administering to a subject in need thereof, a therapeutically effective amount of a GABA_{A} agonist, or a pharmaceutically acceptable salt thereof, and a α1β2/3γ2 GABA inhibitor, or a pharmaceutically acceptable salt thereof, in an amount effective to inhibit an adverse effect mediated by the GABA_{A} agonist.

24. The method of claim 23, wherein the administration of the GABA_{A} agonist and the α1β2/3γ2 GABA inhibitor treats the pain, epilepsy, or depression with a greater therapeutic window than administration of the GABA_{A} agonist alone in the treatment of pain, epilepsy, or depression.

25. The method of claim 23 or 24, wherein the adverse effect is selected from the group consisting of tolerance to a therapeutic effect of the GABA_{A} agonist, addiction, drowsiness, ataxia, sedation, and amnesia in the subject.

26. The method of any of claims 23-25, wherein the disorder is pain.

27. The method of any of claims 23-25, wherein the disorder is inflammatory pain, neuropathic pain, or nociceptive pain.

28. The method of any of claims 23-25, wherein the disorder is epilepsy.

29. The method of any of claims 23-25, wherein the disorder is depression.

30. A method of inhibiting an adverse effect of a GABA_{A} agonist, the adverse effect being selected from the group consisting of tolerance to antinociception, drowsiness, and addiction, comprising administering to a subject in need thereof, an effective amount of a α1β2/3γ2 GABA inhibitor.

31. The method of claim 30, wherein the adverse effect is tolerance to antinociception, comprising administering a tolerance-inhibiting amount of the α1β2/3γ2 GABA inhibitor.

32. The method of any of claims 23-31, wherein the GABA_{A} agonist is the GABA_{A} agonist of any of claims 10-14.

33. The method of any of claims 23-32, wherein the α1β2/3γ2 GABA inhibitor is the α1β2/3γ2 GABA inhibitor of any of claims 15-18.

34. Use of a pharmaceutical combination of a GABA_{A} agonist, or a pharmaceutically acceptable salt thereof, and a α1β2/3γ2 GABA inhibitor, or a pharmaceutically acceptable salt thereof, for the preparation of a medicament for the treatment of pain, epilepsy, or depression.

35. The use of claim 34, wherein the treatment has a reduced GABA_{A} agonist-mediated adverse effect compared to use of the GABA_{A} agonist alone in the treatment of pain, epilepsy, or depression.

36. The use of claim 34 or 35, wherein the use is simultaneous, separate, or sequential.

37. The use of any of claims 34-36 comprising use of a therapeutically effective amount of the GABA_{A} agonist, or a pharmaceutically acceptable salt thereof, and an amount of the α1β2/3γ2 GABA inhibitor, or a pharmaceutically acceptable salt thereof, effective to inhibit an adverse effect mediated by the GABA_{A} agonist.

38. The use of any of claims 35-37, wherein the adverse effect is drowsiness, sedation, ataxia, amnesia, addiction, or tolerance to a therapeutic effect of the GABA_{A} agonist in a subject.

39. The use of any of claims 34-38, wherein the GABA_{A} agonist is the GABA_{A} agonist of any of claims 10-14.

40. The use of any of claims 34-39, wherein the α1β2/3γ2 GABA inhibitor is the α1β2/3γ2 GABA inhibitor of any of claims 15-18.

41. Use of a α1β2/3γ2 GABA inhibitor, or a pharmaceutically acceptable salt thereof, for the preparation of a medicament for the inhibition of an adverse effect mediated by a GABA_{A} agonist, the adverse effect being selected from the group consisting of tolerance to antinociception, drowsiness, and addiction.

42. Use of a α1β2/3γ2 GABA inhibitor, or a pharmaceutically acceptable salt thereof, for the preparation of a medicament for the inhibition of tolerance to an antinociceptive effect of a GAB AA agonist.

43. The use of claim 41 or 42, wherein the GABA_{A} agonist is the GABA_{A} agonist of any of claims 10-14.

44. The use of any of claims 41-43, wherein the α1β2/3γ2 GABA inhibitor is the α1β2/3γ2 GABA inhibitor of any of claims 15-18.

45. A pharmaceutical composition comprising
a) a GABA_{A} agonist, or a pharmaceutically acceptable salt thereof;
b) a α1β2/3γ2 GABA inhibitor, or a pharmaceutically acceptable salt thereof; and
c) a pharmaceutically acceptable carrier.

46. The pharmaceutical composition of claim 45, Wherein the GABA_{A} agonist is the GABA_{A} agonist of any of claims 10-14.

47. The pharmaceutical composition of claim 45 or 46, wherein the α1β2/3γ2 GABA inhibitor is the α1β2/3γ2 GABA inhibitor of any of claims 15-18.

48. A kit comprising
a) a GABA_{A} agonist, or a pharmaceutically acceptable salt thereof;
b) a α1β2/3γ2 GABA inhibitor, or a pharmaceutically acceptable salt thereof; and
c) instructions for use.

49. The kit of claim 48, wherein the GABA_{A} agonist is the GABA_{A} agonist of any of claims 10-14.

50. The kit of claim 48 or 49, wherein the α1β2/3γ2 GABA inhibitor is the α1β2/3γ2 GABA inhibitor of any of claims 15-18.

51. A pharmaceutical combination comprising a GABA_{A} agonist, or a pharmaceutically acceptable salt thereof, and a α1β2/3γ2 GABA inhibitor, or a pharmaceutically acceptable salt thereof, for use as a medicament.

52. The pharmaceutical combination for use of claim 51, wherein the GABA_{A} agonist is the GABA_{A} agonist of any of claims 10-14.

53. The pharmaceutical combination for use of claim 51 or 52, wherein the α1β2/3γ2 GABA inhibitor is the α1β2/3γ2 GABA inhibitor of any of claims 15-18.
